(19) United States
(12) Patent Application Publication
(10) Pub. No.: US 2003/0119100 A1
(43) Pub. Date: Jun. 26, 2003

(54) DNA molecules encoding human nuclear receptor proteins

(75) Inventor: Fang Chen, North Wales, PA (US)

Correspondence Address: MERCK AND CO INC, P O BOX 2000, RAHWAY, NJ, 070650907

(73) Assignee: Merck & Co., Inc., Rahway, NJ

(21) Appl. No.: 10/054,841

(22) Filed: Jan. 23, 2002

Related U.S. Application Data
(63) Continuation of application No. 09/487,379, filed on Jan. 18, 2000, now abandoned.
Application No. 09/487,379 is a division of application No. 09/141,000, filed on Aug. 26, 1998, now Pat. No. 6,054,295.
(60) Provisional application No. 60/078,633, filed on Mar. 19, 1998.
(60) Provisional application No. 60/062,902, filed on Oct. 21, 1997.
(60) Provisional application No. 60/057,090, filed on Aug. 27, 1997.

(51) Int. Cl. C07K 14/705; C07H 21/04; C12P 21/02; C12N 5/06; C12N 15/87

(52) U.S. Cl. 435/69.1; 435/455; 435/325; 435/320.1; 536/23.5; 530/350

(57) ABSTRACT

The present invention discloses the isolation and characterization of cDNA molecules encoding two human nuclear receptor proteins, designated nNR1, nNR2 and/or nNR2-1. Also within the scope of the disclosure are recombinant vectors, recombinant host cells, methods of screening for modulators of nNR1, nNR2 and/or nNR2-1 activity, and production of antibodies against nNR1, nNR2 and/or nNR2-1, or epitopes thereof.

### CROSS-REFERENCE TO RELATED APPLICATIONS

This application is a continuation of U.S. Provisional Application Serial No. 60/078,633, filed Mar. 19, 1998 which is a continuation-in-part of U.S. Provisional Application Serial No. 60/062,902, filed Oct. 21, 1997, which is a continuation-in-part of U.S. Provisional Application Serial No. 60/057,090, filed Aug. 27, 1997.

### STATEMENT REGARDING FEDERALLY-SPONSORED R&D

Not applicable

### REFERENCE TO MICROFICHE APPENDIX

Not applicable.

### FIELD OF THE INVENTION

The present invention relates in part to isolated nucleic acid molecules (polynucleotide) which encode human nuclear receptor proteins, referred to throughout as nNR1, nNR2 and/or nNR2-1. The present invention also relates to recombinant vectors and recombinant hosts which contain a DNA fragment encoding nNR1, nNR2 and/or nNR2-1, substantially purified forms of associated human nNR1, nNR2 and/or nNR2-1 protein, human mutant proteins, and methods associated with identifying compounds which modulate nNR1, nNR2 and/or nNR2-1 activity.

### BACKGROUND OF THE INVENTION

The nuclear receptor superfamily, which includes steroid hormone receptors, are small chemical ligand-inducible transcription factors which have been shown to play roles in controlling development, differentiation and physiological function. Isolation of cDNA clones encoding nuclear receptors reveal several characteristics. First, the NH₂-terminal regions, which vary in length between receptors, is hypervariable with low homology between family members. There are three internal regions of conservation, referred to as domain I, II and III. Region I is a cysteine-rich region which is referred to as the DNA binding domain (DBD). Regions II and III are within the COOH-terminal region of the protein and is also referred to as the ligand binding domain (LBD). For a review, see Power et al. (1992, Trends in Pharmaceutical Sciences 13: 318-323).

The lipophilic hormones that activate steroid receptors are known to be associated with human diseases. Therefore, the respective nuclear receptors have been identified as possible targets for therapeutic intervention. For a review of the mechanism of action of various steroid hormone receptors, see Tsai and O'Malley (1994, Annu. Rev. Biochem. 63: 451-486).

Recent work with non-steroid nuclear receptors has also shown the potential as drug targets for therapeutic intervention. This work reports that peroxisome proliferator activated receptor g (PPARg), identified by a conserved DBD region, promotes adipocyte differentiation upon activation and that thiazolidinediones, a class of antidiabetic drugs, function through PPARg (Tontonoz et al., 1994, Cell 79: 1147-1156; Lehmann et al., 1995, J. Biol. Chem. 270(22): 12953-12956; Teboul et al., 1995, J. Biol. Chem. 270(47): 28183-28187). This indicates that PPARg plays a role in glucose homeostasis and lipid metabolism.

Giguère, et al. (1988, Nature 331: 91-94) isolated two cDNAs which encode a human nuclear receptor, referred to as hERR1 and hEER2. The authors did not assign a ligand and subsequent ligand-inducible function to either of these human nuclear receptors.

Trapp and Holsboer (1996, J. Biol. Chem. 271(17): 9879-9882) show that hERR2 acts as a cell-specific inhibitor of glucocorticoid receptor-mediated gene expression.

It would be advantageous to identify a gene encoding an additional human nuclear receptor protein. A nucleic acid molecule expressing a human nuclear receptor protein will be useful in screening for compounds acting as a modulator of cell differentiation, cell development and physiological function. The present invention addresses and meets these needs by disclosing isolated nucleic acid molecules which express a human nuclear receptor protein which will have a role in cell differentiation and development.

### SUMMARY OF THE INVENTION

The present invention relates to isolated nucleic acid molecules (polynucleotides) which encode novel nuclear receptor proteins, preferably human nuclear receptor proteins, such as human nuclear receptor proteins exemplified and referred to throughout this specification as nNR1, nNR2 and/or nNR2-1.

The present invention also relates to isolated nucleic acid fragments of nNR1 (SEQ ID NO:1) and nNR2 (SEQ ID NO:3) which encode mRNA expressing a biologically active novel human nuclear receptor. Any such nucleic acid fragment will encode either a protein or protein fragment comprising at least an intracellular DNA-binding domain and/or ligand binding domain, domains conserved throughout the human nuclear receptor family domain which exist in nNR1 (SEQ ID NO:2) and nNR2 (SEQ ID NO:4). Any such polynucleotide includes but is not necessarily limited to nucleotide substitutions, deletions, additions, amino-terminal truncations and carboxy-terminal truncations such that these mutations encode mRNA which express a protein or protein fragment of diagnostic, therapeutic or prophylactic use and would be useful for screening for agonists and/or antagonists for nNR1, nNR2 and/or nNR2-1 function.

The isolated nucleic acid molecule of the present invention may include a deoxyribonucleic acid molecule (DNA), such as genomic DNA and complementary DNA (cDNA), which may be single (coding or noncoding strand) or double stranded, as well as synthetic DNA, such as a synthesized, single stranded polynucleotide. The isolated nucleic acid molecule of the present invention may also include a ribonucleic acid molecule (RNA).

The present invention also relates to recombinant vectors and recombinant hosts, both prokaryotic and eukaryotic, which contain the substantially purified nucleic acid molecules disclosed throughout this specification.

A preferred aspect of the present invention is disclosed in FIGS. 1A-C and SEQ ID NO:1, a human cDNA encoding a novel nuclear trans-acting receptor protein, nNR1.

Another preferred aspect of the present invention is disclosed in FIGS. 4A-C and SEQ ID NO:3, a human cDNA encoding a novel nuclear trans-acting receptor protein, nNR2.

Another preferred aspect of the present invention is disclosed in FIGS. 7A-C and SEQ ID NO:5, a human cDNA encoding a truncated version of nNR2, referred to as nNR2-1.

The present invention also relates to a substantially purified form of the novel nuclear trans-acting receptor protein, nNR1, which is disclosed in FIGS. 2A-F and FIG. 3 and as set forth in SEQ ID NO:2.

The present invention also relates to biologically active fragments and/or mutants of nNR1 as set forth as SEQ ID NO:2, including but not necessarily limited to amino acid substitutions, deletions, additions, amino terminal truncations and carboxy-terminal truncations such that these mutations provide for proteins or protein fragments of diagnostic, therapeutic or prophylactic use and would be useful for screening for agonists and/or antagonists of nNR1 function.

The present invention also relates to a substantially purified form of the novel nuclear trans-acting receptor protein, nNR2, which is disclosed in FIGS. 5A-E and FIG. 6 and as set forth in SEQ ID NO:4.

The present invention also relates to biologically active fragments and/or mutants of nNR2 as set forth as SEQ ID NO:4, including but not necessarily limited to amino acid substitutions, deletions, additions, amino terminal truncations and carboxy-terminal truncations such that these mutations provide for proteins or protein fragments of diagnostic, therapeutic or prophylactic use and would be useful for screening for agonists and/or antagonists of nNR2 function.

A preferred aspect of the present invention is disclosed in FIG. 3 and is set forth as SEQ ID NO:2, the amino acid sequence of the novel nuclear trans-acting receptor protein, nNR1.

A preferred aspect of the present invention is disclosed in FIG. 6 and is set forth as SEQ ID NO:4, the amino acid sequence of the novel nuclear trans-acting receptor protein, nNR2.

A preferred aspect of the present invention is disclosed in FIG. 8 and is set forth as SEQ ID NO:6, the amino acid sequence of a truncated version of nNR2, refereed to as nNR2-1.

The present invention also relates to polyclonal and monoclonal antibodies raised in response to either the human form of nNR1, nNR2 and/or nNR2-1 disclosed herein, or a biologically active fragment thereof. It will be especially preferable to raise antibodies against epitopes within the NH₂-terminal domain of nNR1, nNR2 and/or nNR2-1, which show the least homology to other known proteins belonging to the human nuclear receptor superfamily. To this end, the DNA molecules, RNA molecules, recombinant protein and antibodies of the present invention may be used to screen and measure levels of human nNR1, nNR2 and/or nNR2-1. The recombinant proteins, DNA molecules, RNA molecules and antibodies lend themselves to the formulation of kits suitable for the detection and typing of human nNR1, nNR2 and/or nNR2-1.

The present invention also relates to isolated nucleic acid molecules which are fusion constructions expressing fusion proteins useful in assays to identify compounds which modulate wild-type human nNR1, nNR2 and/or nNR2-1 activity. A preferred aspect of this portion of the invention includes, but is not limited to, glutathione S-transferase GST-nNR1 and/or GST-nNR2 fusion constructs. These fusion constructs include, but are not limited to, all or a portion of the ligand-binding domain of nNR1, nNR2 and/or nNR2-1, respectively, as an in-frame fusion at the carboxy terminus of the GST gene. The disclosure of SEQ ID NOS:1-4 allow the artisan of ordinary skill to construct any such nucleic acid molecule encoding a GST-nuclear receptor fusion protein. Soluble recombinant GST-nuclear receptor fusion proteins may be expressed in various expression systems, including Spodoptera frugiperda (Sf21) insect cells (Invitrogen) using a baculovirus expression vector (e.g., Bac-N-Blue DNA from Invitrogen or pAcG2T from Pharmingen).

It is an object of the present invention to provide an isolated nucleic acid molecule which encodes a novel form of a nuclear receptor protein such as human nNR1 and/or human nNR2, human nuclear receptor protein fragments of full length proteins such as nNR1, nNR2 and/or nNR2-1, and mutants which are derivatives of SEQ ID NO:2 and SEQ ID NO:4. Any such polynucleotide includes but is not necessarily limited to nucleotide substitutions, deletions, additions, amino-terminal truncations and carboxy-terminal truncations such that these mutations encode mRNA which express a protein or protein fragment of diagnostic, therapeutic or prophylactic use and would be useful for screening for agonists and/or antagonists for nNR1, nNR2 and/or nNR2-1 function.

It is a further object of the present invention to provide the human nuclear receptor proteins or protein fragments encoded by the nucleic acid molecules referred to in the preceding paragraph.

It is a further object of the present invention to provide recombinant vectors and recombinant host cells which comprise a nucleic acid sequence encoding human nNR1, nNR2 and/or nNR2-1 or a biological equivalent thereof.

It is an object of the present invention to provide a substantially purified form of nNR1, as set forth in SEQ ID NO:2.

It is an object of the present invention to provide for biologically active fragments and/or mutants of nNR1, including but not necessarily limited to amino acid substitutions, deletions, additions, amino terminal truncations and carboxy-terminal truncations such that these mutations provide for proteins or protein fragments of diagnostic, therapeutic or prophylactic use.

It is an object of the present invention to provide a substantially purified form of nNR2, as set forth in SEQ ID NO:4.

It is an object of the present invention to provide for biologically active fragments and/or mutants of nNR2, including but not necessarily limited to amino acid substitutions, deletions, additions, amino terminal truncations and carboxy-terminal truncations such that these mutations provide for proteins or protein fragments of diagnostic, therapeutic or prophylactic use.

It is also an object of the present invention to provide for NNR1- and/or nNR2-based in-frame fusion constructions, methods of expressing these fusion constructions and biological equivalents disclosed herein, related assays, recombinant cells expressing these constructs and agonistic and/or antagonistic compounds identified through the use DNA molecules encoding human nuclear receptor proteins such as nNR1, nNR2 and/or nNR2-1.

As used herein, “DBD” refers to DNA binding domain.

As used herein, “LBD” refers to ligand binding domain.

As used herein, the term “mammalian host” refers to any mammal, including a human being.

### BRIEF DESCRIPTION OF THE DRAWINGS

FIGS. 1A-C shows the nucleotide sequence (SEQ ID NO:1) which comprises the open reading frame encoding the human nuclear receptor protein, nNR1.

FIGS. 2A-F shows the nucleotide sequence of the double stranded cDNA molecule (SEQ ID NO:1 and SEQ ID NO:29) which encodes nNR1, and the amino acid sequence of nNR1 (SEQ ID NO:2). The region in bold and underline is the DNA binding domain.

FIG. 3 shows the amino acid sequence of nNR1 (SEQ ID NO:2). The region in bold and underline is the DNA binding domain.

FIGS. 4A-C shows the nucleotide sequence (SEQ ID NO:3) which comprises the open reading frame encoding the human nuclear receptor protein, nNR2.

FIGS. 5A-E shows the nucleotide sequence of the double stranded cDNA molecule (SEQ ID NO:1 and SEQ ID NO:29) which encodes nNR2, and the amino acid sequence of nNR2 (SEQ ID NO:4). The region in-bold and underline is the DNA binding domain.

FIG. 6 shows the amino acid sequence of nNR2 (SEQ ID NO:4). The region in bold and underline is the DNA binding domain.

FIGS. 7A-C shows the nucleotide sequence (SEQ ID NO:5) which comprises the open reading frame encoding the human nuclear receptor protein, nNR2.

FIG. 8 shows the amino acid sequence of nNR2-1, a carboxy-terminal truncated version of nNR2 (SEQ ID NO:6). The region in bold and underline is the DNA binding domain.

### DETAILED DESCRIPTION OF THE INVENTION

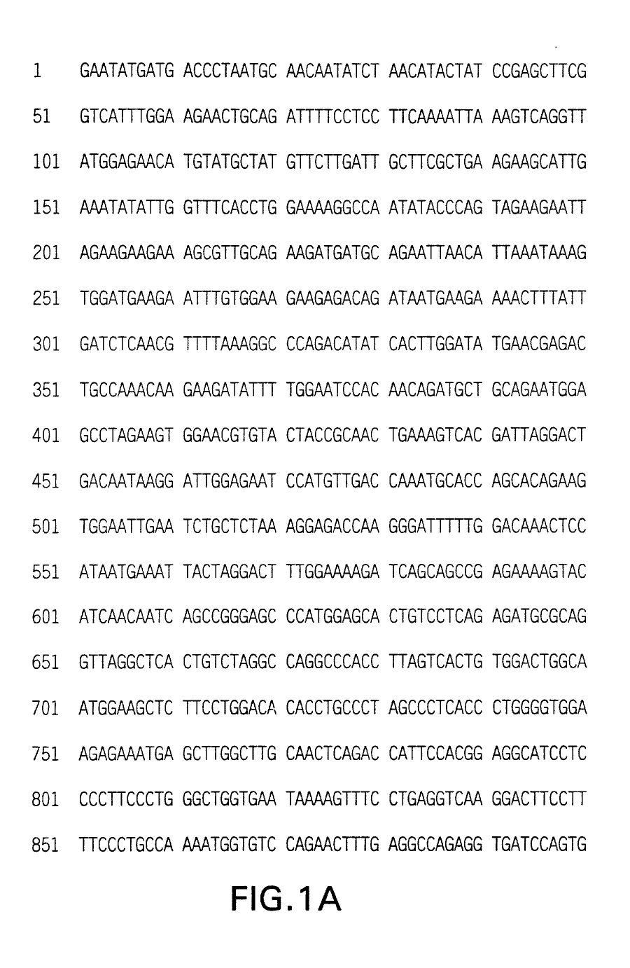

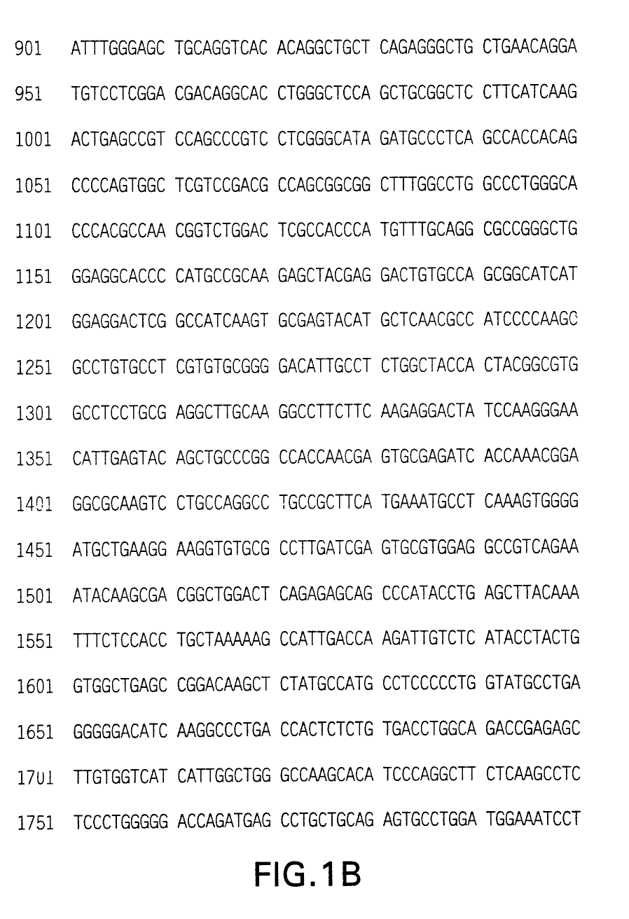

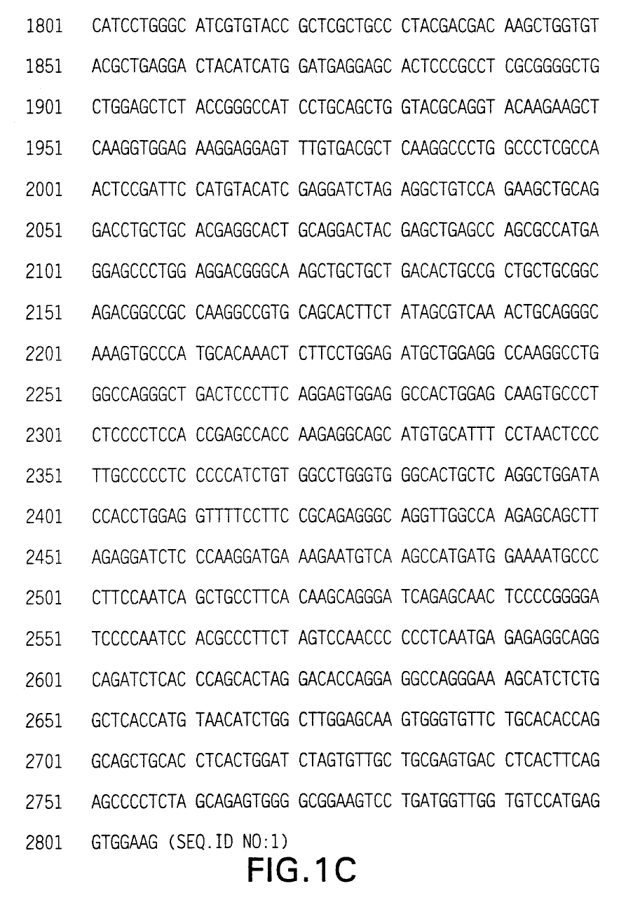

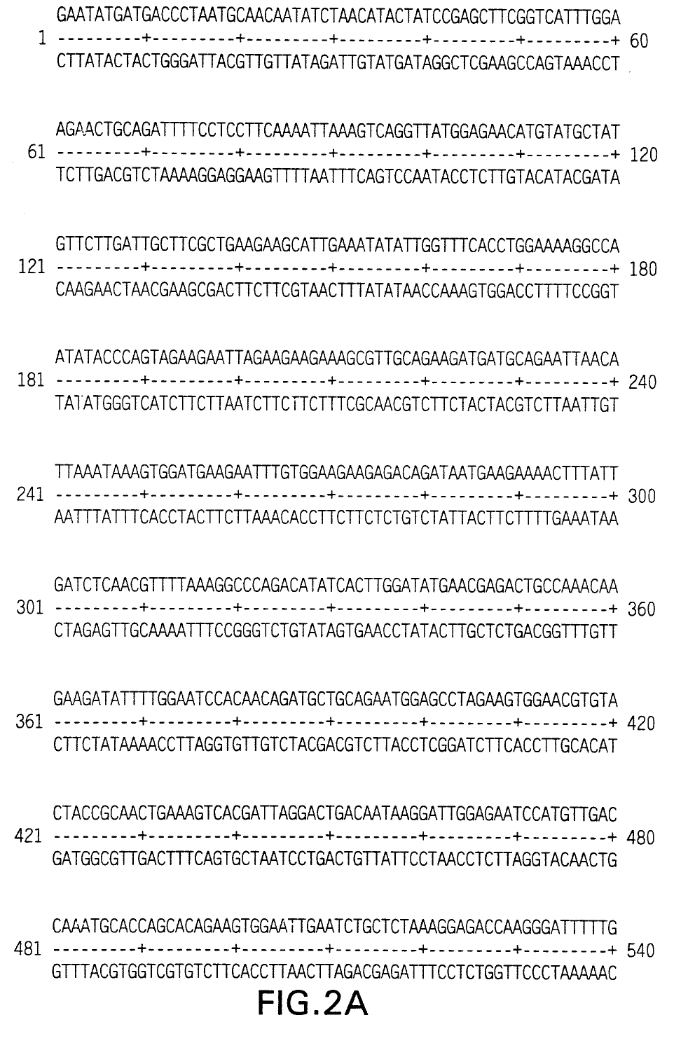

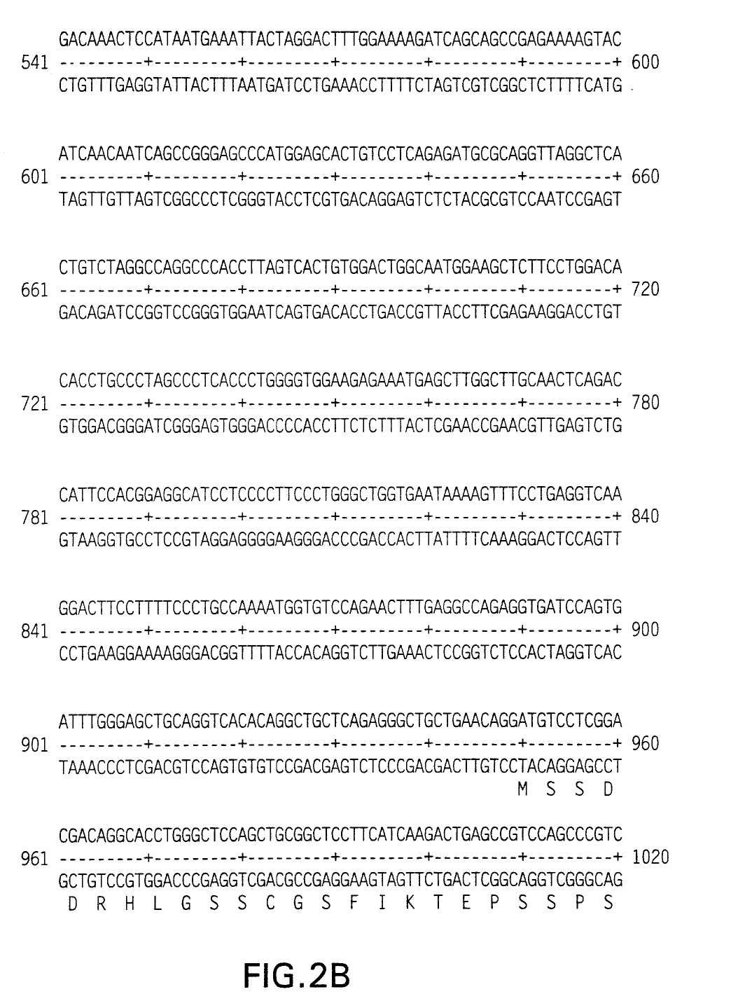

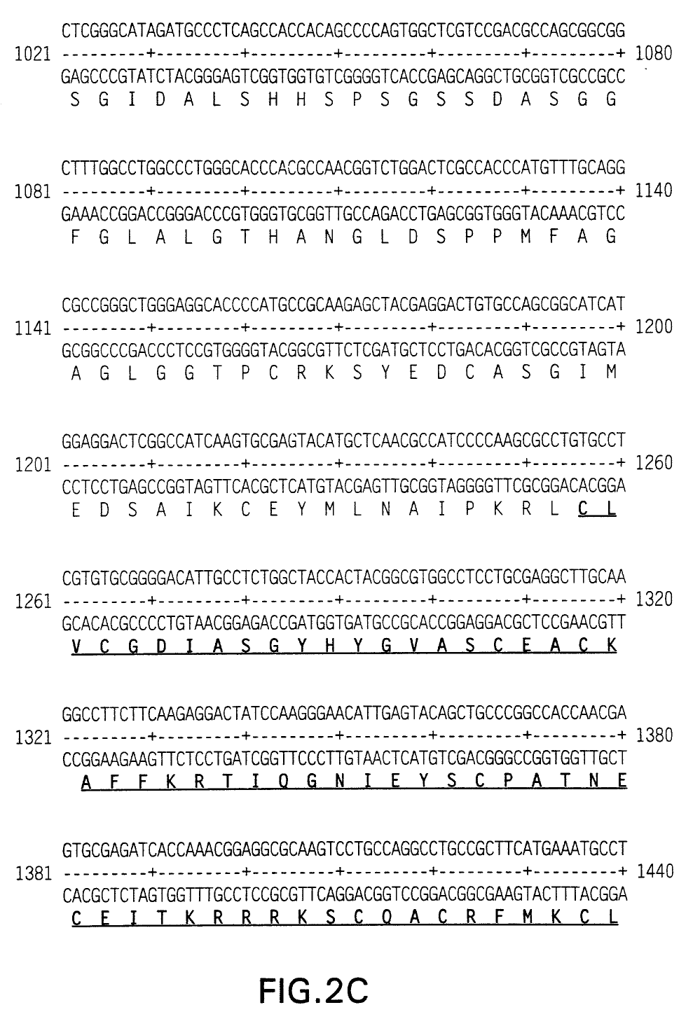

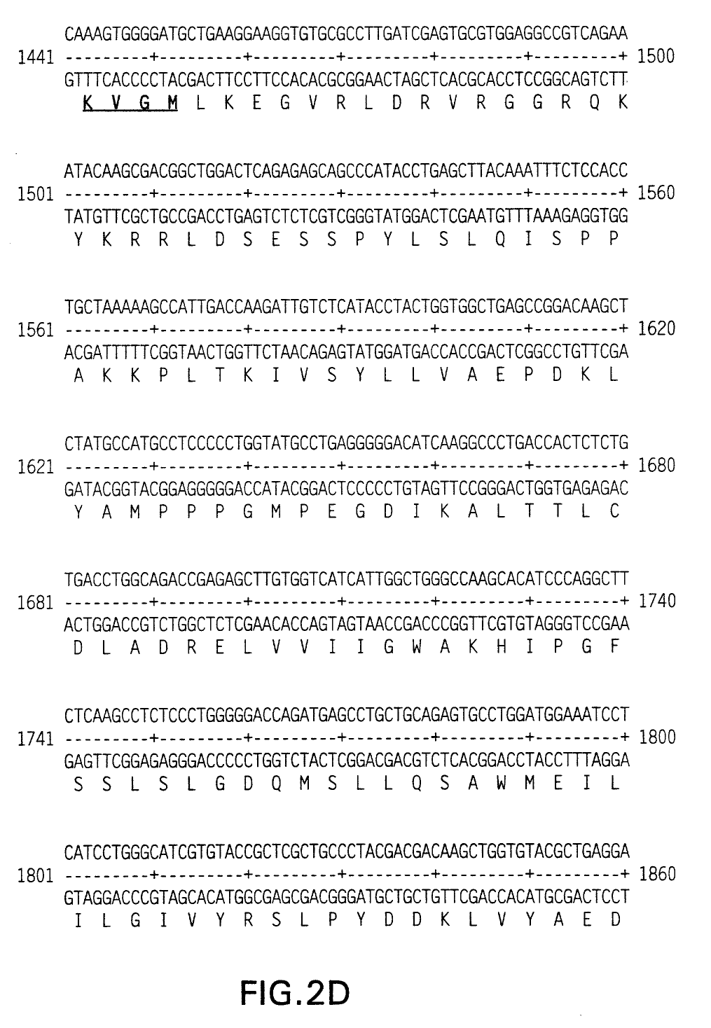

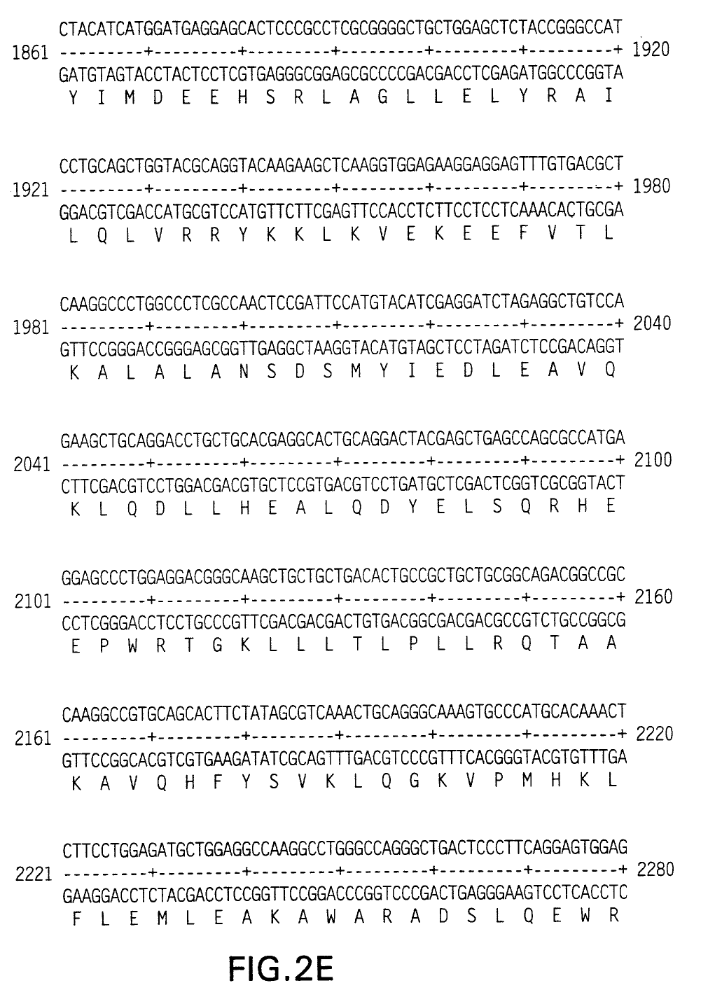

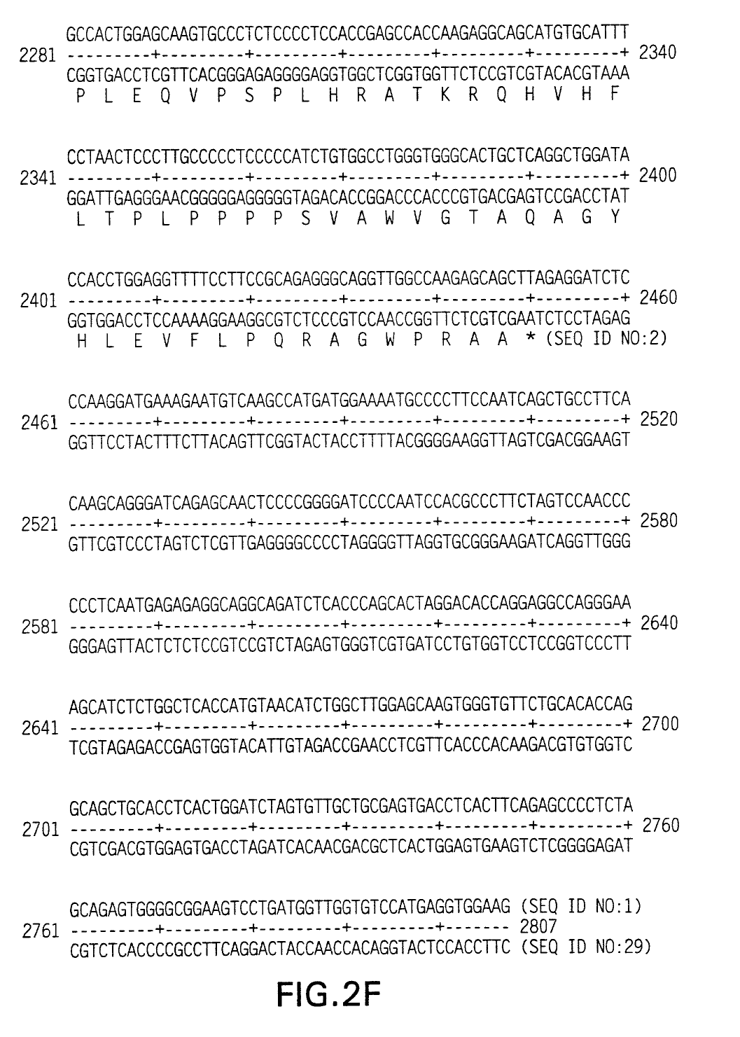

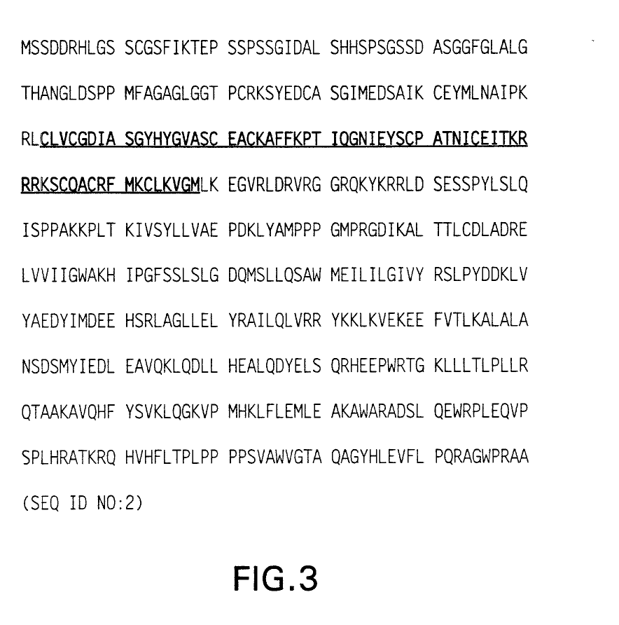

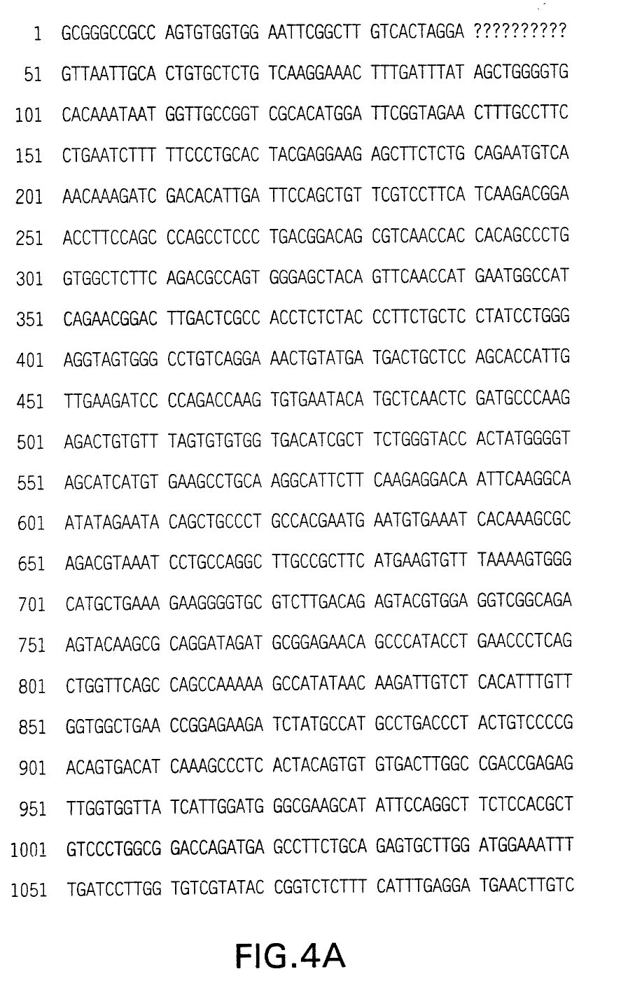

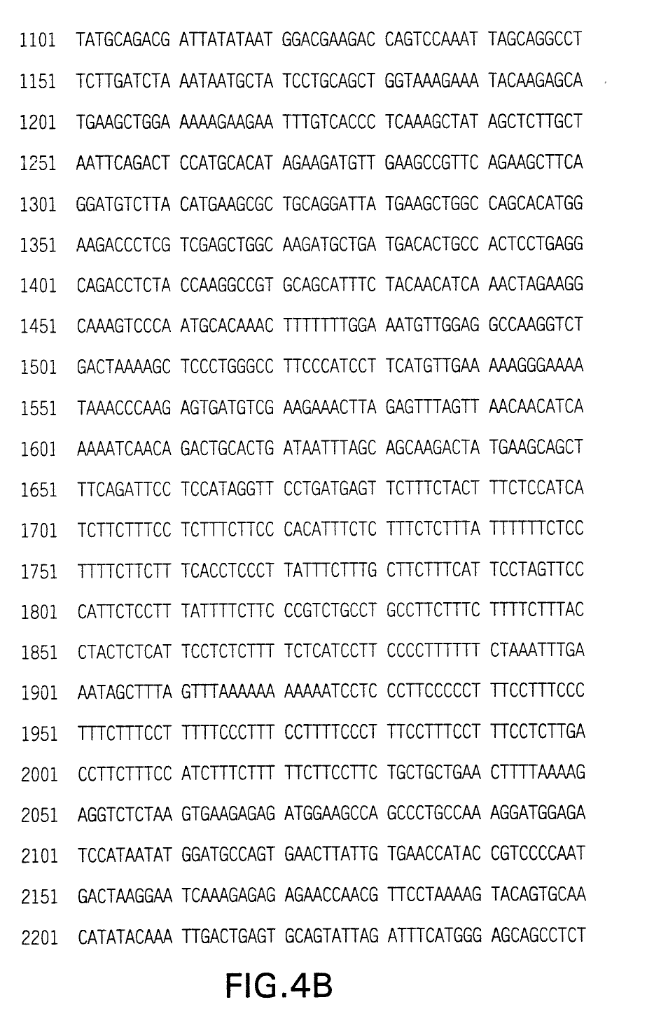

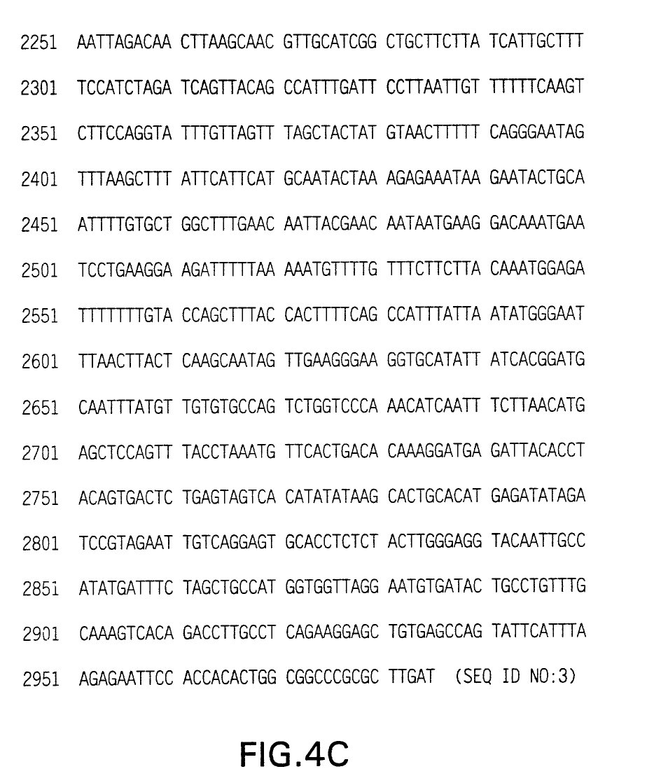

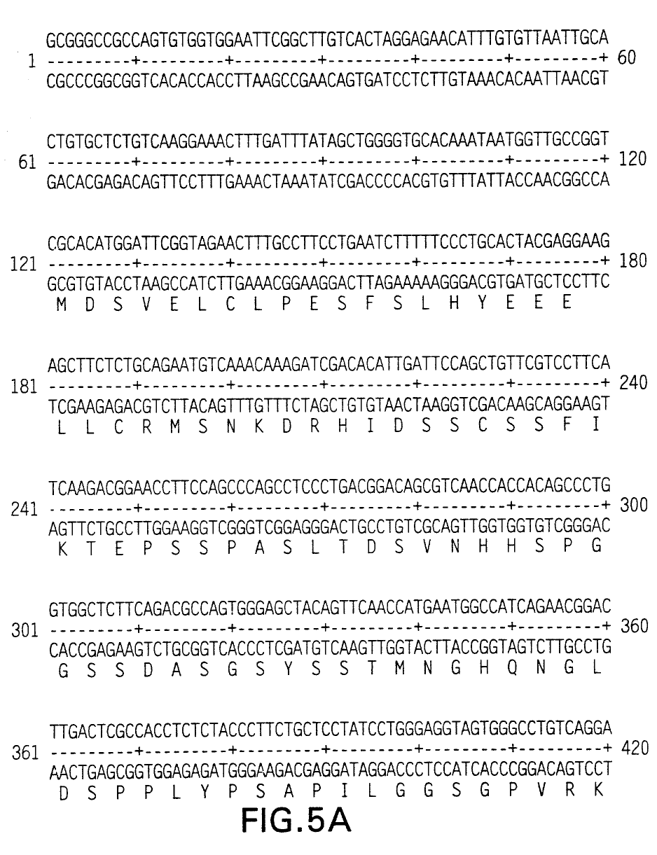

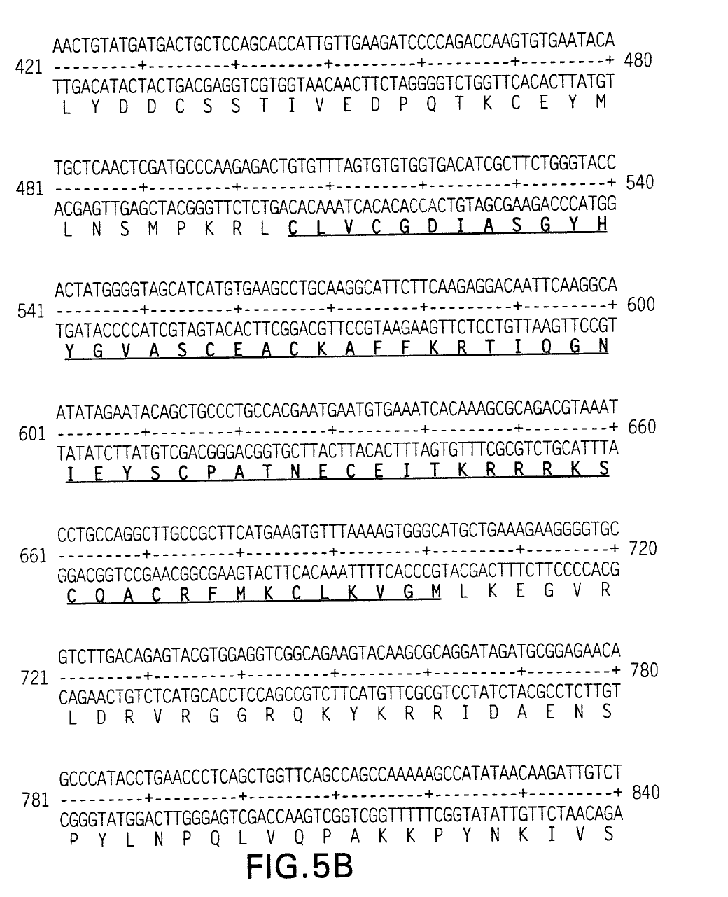

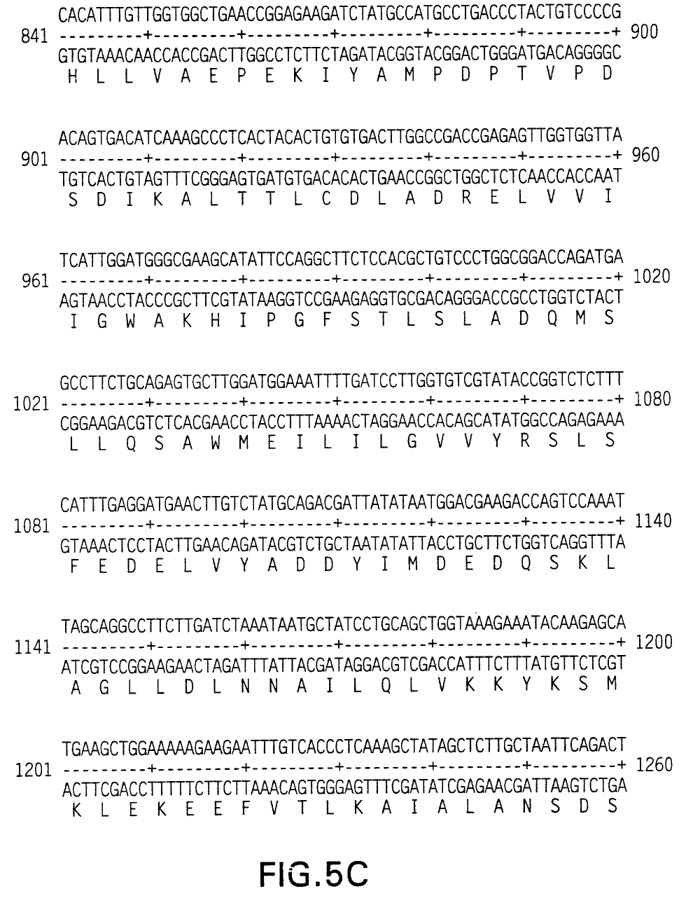

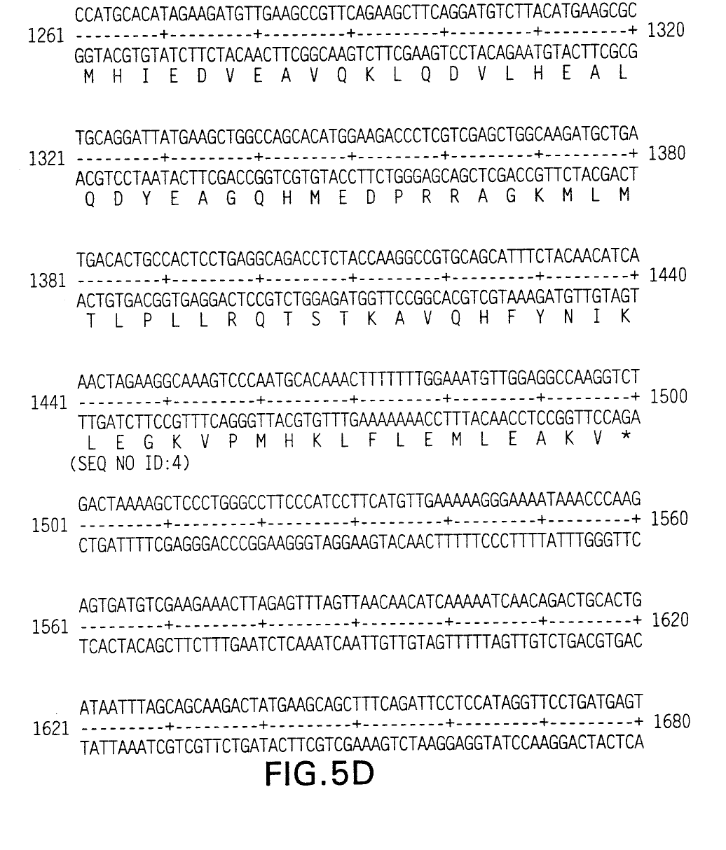

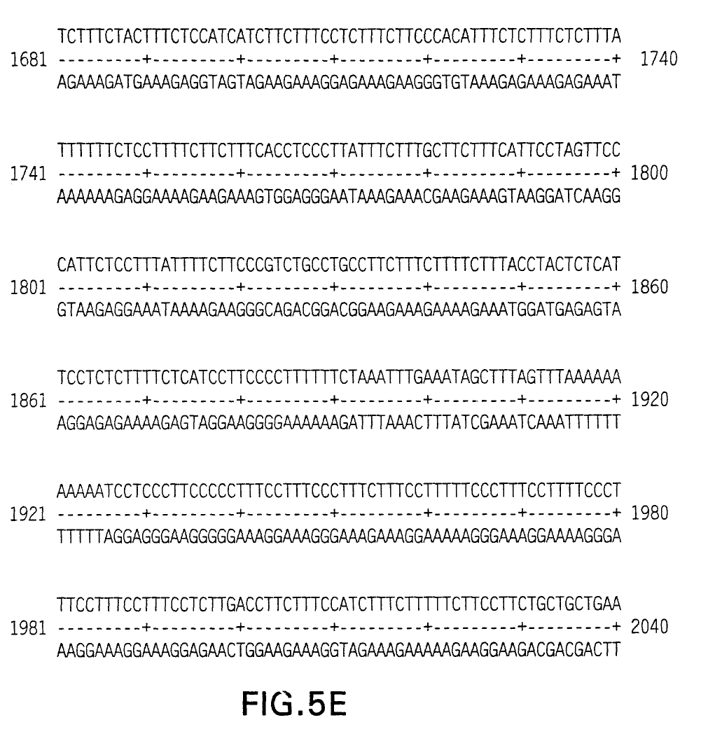

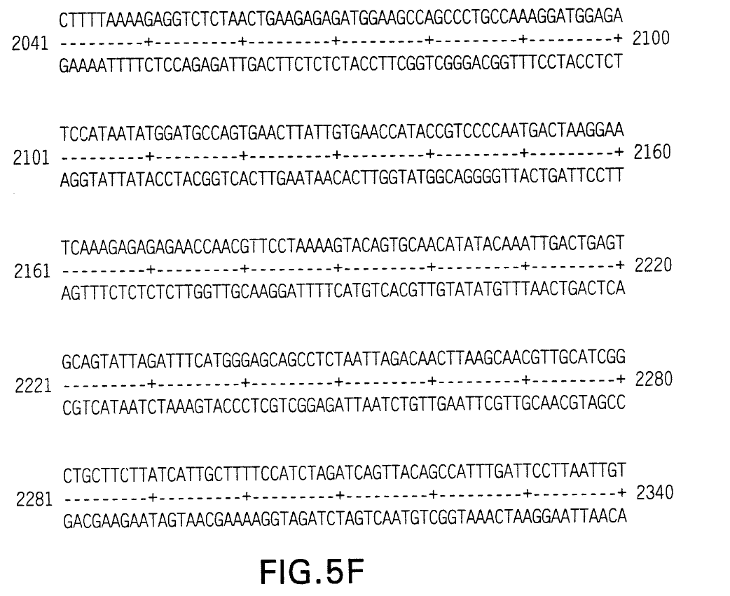

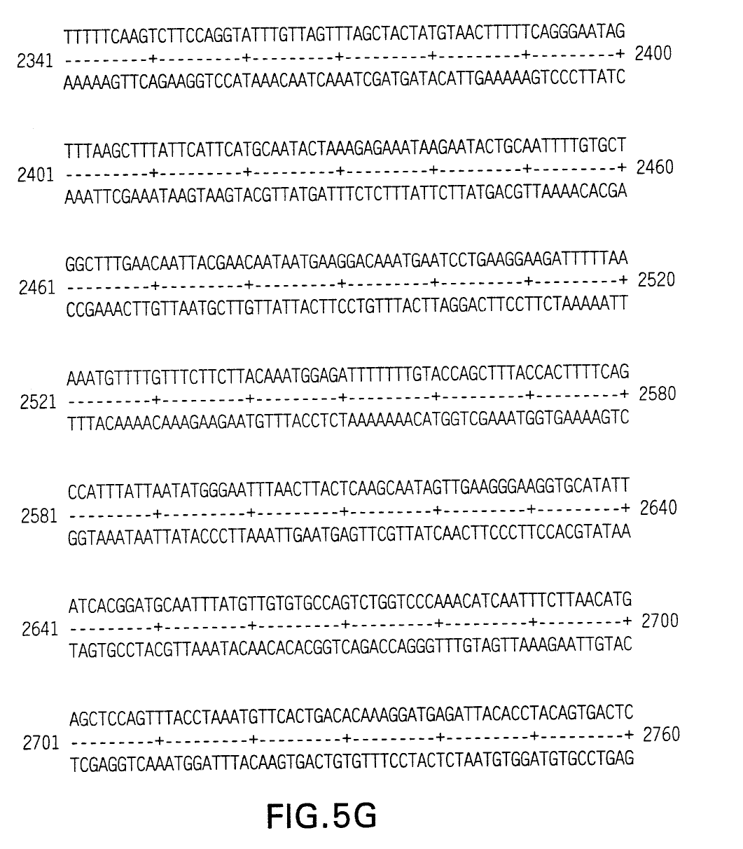

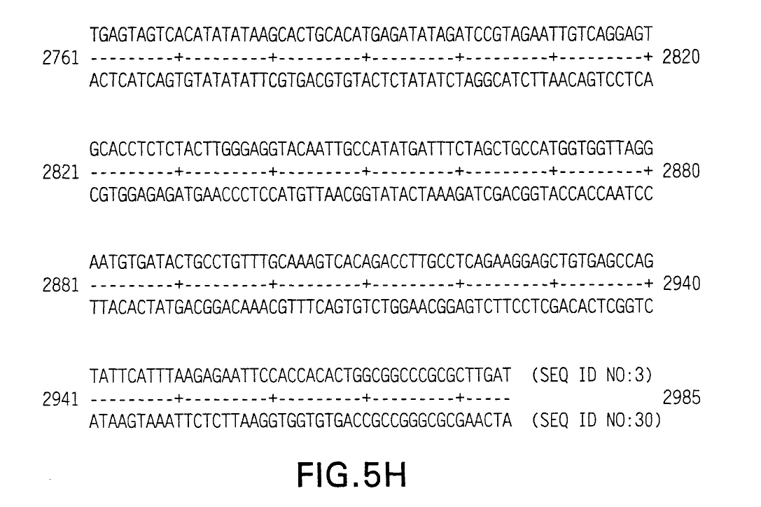

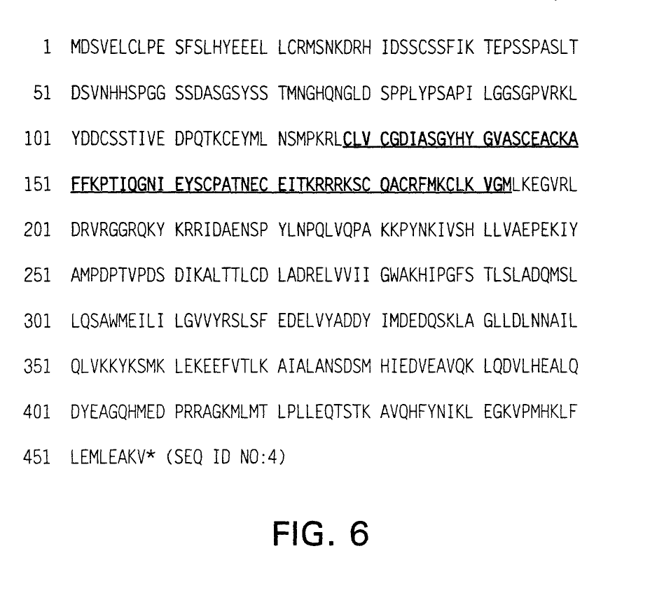

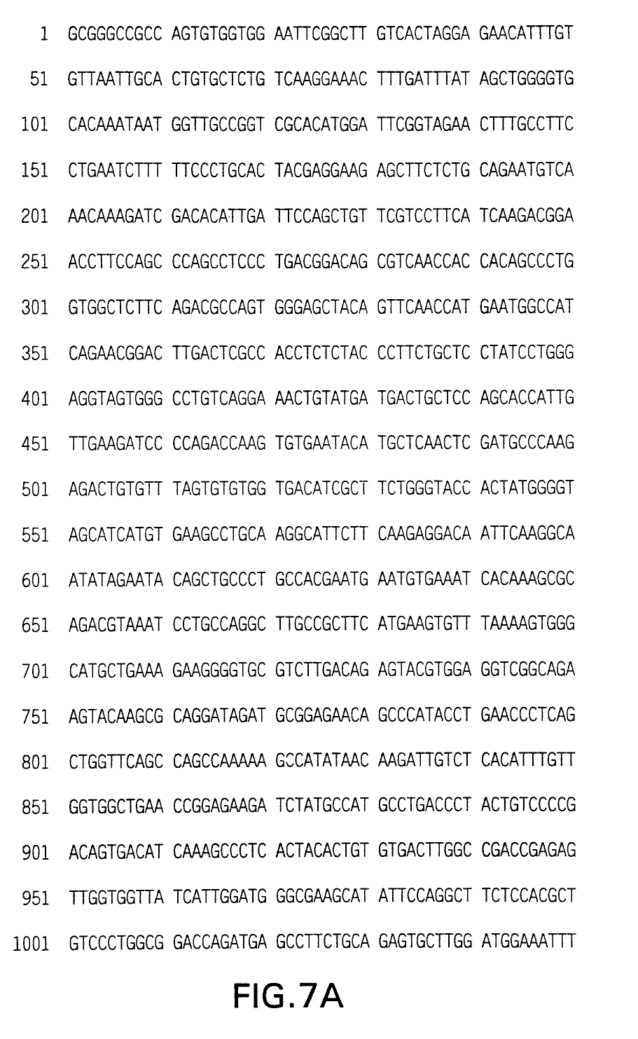

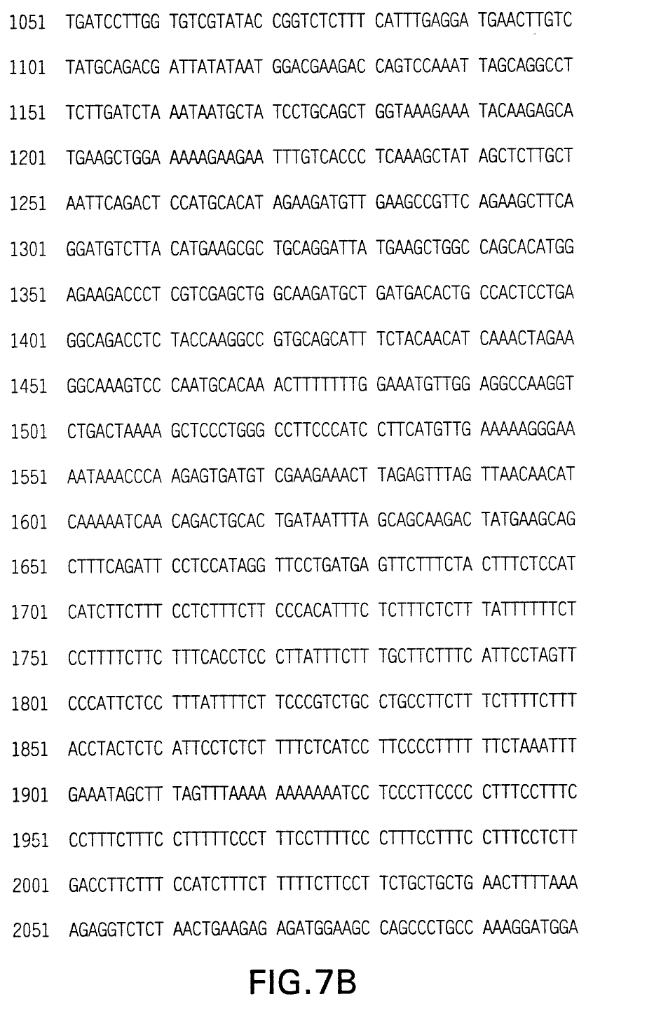

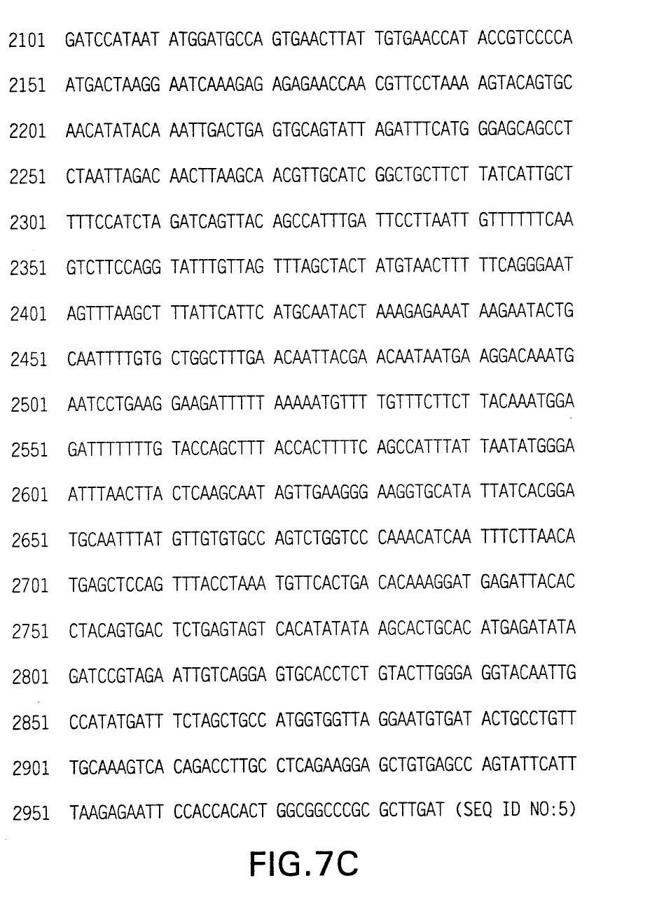

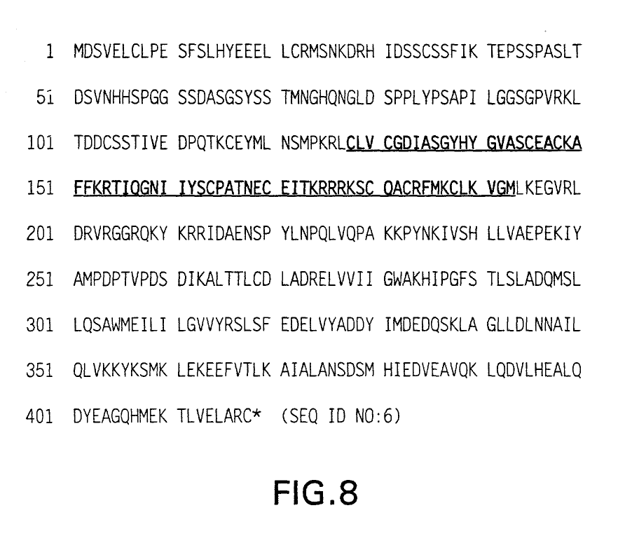

The present invention relates to isolated nucleic acid and protein forms which represent nuclear receptors, preferably but not necessarily limited to human receptors. These expressed proteins are novel nuclear receptors and which are useful in the identification of downstream target genes and ligands regulating their activity. The nuclear receptor superfamily is composed of a group of structurally related receptors which are regulated by chemically distinct ligands. The common structure for a nuclear receptor is a highly conserved DNA binding domain (DBD) located in the center of the peptide and the ligand-binding domain (LBD) at the COOH-terminus. Eight out of the nine non-variant cysteines form two type II zinc fingers which distinguish nuclear receptors from other DNA-binding proteins. The DBDs share at least 50% to 60% amino acid sequence identity even among the most distant members in vertebrates. The superfamily has been expanded within the past decade to contain approximately 25 subfamilies. An EST database search using whole peptide sequences of several representative subfamily members was used to identify two human ESTs (GenBank accession numbers h91890 and w26275 for an EST corresponding to nNR1, nNR2 and/or nNR2-1, respectively). The sequence information from each EST was utilized to isolate and characterize the full length cDNA for the gene corresponding to nNR1 (see FIGS. 1A-C and SEQ ID NO:1) and nNR2 (see FIGS. 4A-C and SEQ ID NO:3). The cDNA of SEQ ID NO:1 encodes nNR1, a protein 500 amino acids in length (FIG. 3; SEQ ID NO:2), which has a distinctive DBD structure (FIGS. 2A-F). The cDNA of SEQ ID NO:3 encodes nNR2, a protein 458 amino acids (FIG. 6; SEQ ID NO:4) in length, and also has a distinctive DBD structure (FIGS. 5A-E). The cDNA of SEQ ID NO:5 encodes nNR2-1, a protein 418 amino acids (FIG. 8; SEQ ID NO:6) in length which is a carboxy terminal truncated version of nNR2. The protein nNR2-1 also has a distinctive DBD structure (FIG. 8).

The nNR1 protein shows 95% homology to hERR2 (Giguère, et al., 1988, Nature 331: 91-94) in the overlapping peptide region. However, nNR1 contains an additional 67 amino acids at the carboxy-terminus in comparison to hERR2. The gene encoding nNR1 is located on locus 14q24.3˜14q31, which is the Alzheimer disease gene 3 (AD3) locus. Therefore, nNR1 may be an endogenous modulator of glucocorticoid receptor (GR) in view of data showing that hERR2 represses GR activity. nNR2 and nNR2-1 share 77% and 75% homology, respectively, at the amino acid level to hERR2 (Giguère, et al., 1988, Nature 331: 91-94) in the overlapping region. The nNR2 and nNR1 proteins show 77% homology at the amino acid level. The gene encoding nNR2 is located on chromosome 1. Both genes are expressed at very low levels in the majority of the tissues examined via RT-PCR.

Therefore, the present invention also relates to isolated nucleic acid fragments of nNR1 (SEQ ID NO:1) and nNR2 (SEQ ID NO:3) which encode mRNA expressing a biologically active novel human nuclear receptor. Any such nucleic acid fragment will encode either a protein or protein fragment comprising at least an intracellular DNA-binding domain and/or ligand binding domain, domains conserved throughout the human nuclear receptor family domain which exist in nNR1 (SEQ ID NO:2) and nNR2 (SEQ ID NO:4). Any such polynucleotide includes but is not necessarily limited to nucleotide substitutions, deletions, additions, amino-terminal truncations and carboxy-terminal truncations such that these mutations encode mRNA which express a protein or protein fragment of diagnostic, therapeutic or prophylactic use and would be useful for screening for agonists and/or antagonists for nNR1, nNR2 and/or nNR2-1 function. Such a nucleic acid fragment is exemplified as an altered version of the DNA fragment encoding nNR2. This DNA molecule (as set forth in SEQ ID NO:5) is identical to SEQ ID NO:3 save for a two nucleotide insertion at nucleotide 1352 of SEQ ID NO:3. This insertion results in a shifted reading frame and introduction of a TGA termination codon 33 nucleotides from the insertion site, resulting in an open reading frame which encodes the carboxy-truncated nNR2 protein, nNR2-1, as shown in FIG. 8 and SEQ ID NO: 6.

The isolated nucleic acid molecule of the present invention may include a deoxyribonucleic acid molecule (DNA), such as genomic DNA and complementary DNA (cDNA), which may be single (coding or noncoding strand) or double stranded, as well as synthetic DNA, such as a synthesized, single stranded polynucleotide. The isolated nucleic acid molecule of the present invention may also include a ribonucleic acid molecule (RNA).

The present invention also relates to recombinant vectors and recombinant hosts, both prokaryotic and eukaryotic, which contain the substantially purified nucleic acid molecules disclosed throughout this specification.

A preferred aspect of the present invention is disclosed in FIGS. 1A-C and SEQ ID NO:1, a human cDNA encoding a novel nuclear trans-acting receptor protein, nNR1, disclosed as follows:

| | |
|---|---|
| GAATATGATG ACCCTAATGC AACAATATCT AACATACTAT CCGAGCTTCG | (SEQ ID NO:1) |
| GTCATTTGGA AGAACTGCAG ATTTTCCTCC TTCAAAATTA AAGTCAGGTT | |
| ATGGAGAACA TGTATGCTAT GTTCTTGATT GCTTCGCTGA AGAAGCATTG | |
| AAATATATTG GTTTCACCTG GAAAAGGCCA ATATACCCAG TAGAAGAATT | |
| AGAAGAAGAA AGCGTTGCAG AAGATGATGC AGAATTAACA TTAAATAAAG | |
| TGGATGAAGA ATTTGTGGAA GAAGAGACAG ATAATGAAGA AAACTTTATT | |
| GATCTCAACG TTTTAAAGGC CCAGACATAT CACTTGGATA TGAACGAGAC | |
| TGCCAAACAA GAAGATATTT TGGAATCCAC AACAGATGCT GCAGAATGGA | |
| GCCTAGAAGT GGAACGTGTA CTACCGCAAC TGAAAGTCAC GATTAGGACT | |
| GACAATAAGG ATTGGAGAAT CCATGTTGAC CAAATGCACC AGCACAGAAG | |
| TGGAATTGAA TCTGCTCTAA AGGAGACCAA GGGATTTTTG GACAAACTCC | |
| ATAATGAAAT TACTAGGACT TTGGAAAAGA TCAGCAGCCG AGAAAAGTAC | |
| ATCAACAATC AGCCGGGAGC CCATGGAGCA CTGTCCTCAG AGATGCGCAG | |
| GTTAGGCTCA CTGTCTAGGC CAGGCCCACC TTAGTCACTG TGGACTGGCA | |
| ATGGAAGCTC TTCCTGGACA CACCTGCCCT AGCCCTCACC CTGGGGTGGA | |
| AGAGAAATGA GCTTGGCTTG CAACTCAGAC CATTCCACGG AGGCATCCTC | |
| CCCTTCCCTG GGCTGGTGAA TAAAAGTTTC CTGAGGTCAA GGACTTCCTT | |
| TTCCCTGCCA AAATGGTGTC CAGAACTTTG AGGCCAGAGG TGATCCAGTG | |
| ATTTGGGAGC TGCAGGTCAC ACAGGCTGCT CAGAGGGCTG CTGAACAGGA | |
| TGTCCTCGGA CGACAGGCAC CTGGGCTCCA GCTGCGGCTC CTTCATCAAG | |
| ACTGAGCCGT CCAGCCCGTC CTCGGGCATA GATGCCCTCA GCCACCACAG | |
| CCCCAGTGGC TCGTCCGACG CCAGCGGCGG CTTTGGCCTG GCCCTGGGCA | |
| CCCACGCCAA CGGTCTGGAC TCGCCACCCA TGTTTGCAGG CGCCGGGCTG | |
| GGAGGCACCC CATGCCGCAA GAGCTACGAG GACTGTGCCA GCGGCATCAT | |
| GGAGGACTCG GCCATCAAGT GCGAGTACAT GCTCAACGCC ATCCCCAAGC | |
| GCCTGTGCCT CGTGTGCGGG GACATTGCCT CTGGCTACCA CTACGGCGTG | |
| GCCTCCTGCG AGGCTTGCAA GGCCTTCTTC AAGAGGACTA TCCAAGGGAA | |
| CATTGAGTAC AGCTGCCCGG CCACCAACGA GTGCGAGATC ACCAAACGGA | |
| GGCGCAAGTC CTGCCAGGCC TGCCGCTTCA TGAAATGCCT CAAAGTGGGG | |
| ATGCTGAAGG AAGGTGTGCG CCTTGATCGA GTGCGTGGAG GCCGTCAGAA | |
| ATACAAGCGA CGGCTGGACT CAGAGAGCAG CCCATACCTG AGCTTACAAA | |
| TTTCTCCACC TGCTAAAAAG CCATTGACCA AGATTGTCTC ATACCTACTG | |
| GTGGCTGAGC CGGACAAGCT CTATGCCATG CCTCCCCCTG GTATGCCTGA | |
| GGGGGACATC AAGGCCCTGA CCACTCTCTG TGACCTGGCA GACCGAGAGC | |
| TTGTGGTCAT CATTGGCTGG GCCAAGCACA TCCCAGGCTT CTCAAGCCTC | |
| TCCCTGGGGG ACCAGATGAG CCTGCTGCAG AGTGCCTGGA TGGAAATCCT | |
| CATCCTGGGC ATCGTGTACC GCTCGCTGCC CTACGACGAC AAGCTGGTGT | |
| ACGCTGAGGA CTACATCATG GATGAGGAGC ACTCCCGCCT CGCGGGGCTG | |
| CTGGAGCTCT ACCGGGCCAT CCTGCAGCTG GTACGCAGGT ACAAGAAGCT | |
| CAAGGTGGAG AAGGAGGAGT TTGTGACGCT CAAGGCCCTG GCCCTCGCCA | |
| ACTCCGATTC CATGTACATC GAGGATCTAG AGGCTCTCCA GAAGCTGCAG | |
| GACCTGCTGC ACGAGGCACT GCAGGACTAC GAGCTGAGCC AGCGCCATGA | |
| GGAGCCCTGG AGGACGGGCA AGCTGCTGCT GACACTGCCG CTGCTGCGGC | |
| AGACGGCCGC CAAGGCCGTG CAGCACTTCT ATAGCGTCAA ACTGCAGGGC | |
| AAAGTGCCCA TGCACAAACT CTTCCTGGAG ATGCTGGAGG CCAAGGCCTG | |
| GGCCAGGGCT GACTCCCTTC AGGAGTGGAG GCCACTGGAG CAAGTGCCCT | |
| CTCCCCTCCA CCGAGCCACC AAGAGGCAGC ATGTGCATTT CCTAACTCCC | |
| TTGCCCCCTC CCCCATCTGT GGCCTGGGTG GGCACTGCTC AGGCTGGATA | |
| CCACCTGGAG GTTTTCCTTC CGCAGAGGGC AGGTTGGCCA AGAGCAGCTT | |
| AGAGGATCTC CCAAGGATGA AAGAATGTCA AGCCATGATG GAAAATGCCC | |
| CTTCCAATCA GCTGCCTTCA CAAGCAGGGA TCAGAGCAAC TCCCCGGGGA | |
| TCCCCAATCC ACGCCCTTCT AGTCCAACCC CCCTCAATGA GAGAGGCAGG | |
| CAGATCTCAC CCAGCACTAG GACACCAGGA GGCCAGGGAA AGCATCTCTG | |
| GCTCACCATG TAACATCTGG CTTGGAGCAA GTGGGTGTTC TGCACACCAG | |
| GCAGCTGCAC CTCACTGGAT CTAGTGTTGC TGCGAGTGAC CTCACTTCAG | |
| AGCCCCTCTA GCAGAGTGGG GCGGAAGTCC TGATGGTTGG TGTCCATGAG | |
| GTGGAAG. | |

Another preferred aspect of the present invention is disclosed in FIGS. 4A-C and SEQ ID NO:3, a human cDNA encoding a novel nuclear trans-acting receptor protein, nNR2, disclosed as follows:

| | |
|---|---|
| GCGGGCCGCC AGTGTGGTGG AATTCGGCTT GTCACTAGGA GAACATTTCT | (SEQ ID NO:3) |
| GTTAATTGCA CTGTGCTCTG TCAAGGAAAC TTTGATTTAT AGCTGGGGTG | |
| CACAAATAAT GGTTGCCGGT CGCACATGGA TTCGGTAGAA CTTTGCCTTC | |
| CTGAATCTTT TTCCCTGCAC TACGAGGAAG AGCTTCTCTG CAGAATGTCA | |
| AACAAAGATC GACACATTGA TTCCAGCTGT TCGTCCTTCA TCAAGACGGA | |
| ACCTTCCAGC CCAGCCTCCC TGACGGACAG CGTCAACCAC CACAGCCCTG | |
| GTGGCTCTTC AGACGCCAGT GGGAGCTACA GTTCAACCAT GAATGGCCAT | |
| CAGAACGGAC TTGACTCGCC ACCTCTCTAC CCTTCTGCTC CTATCCTGGG | |
| AGGTAGTGGG CCTGTCAGGA AACTGTATGA TGACTGCTCC AGCACCATTG | |
| TTGAAGATCC CCAGACCAAG TGTGAATACA TGCTCAACTC GATGCCCAAG | |
| AGACTGTGTT TAGTGTGTGG TGACATCGCT TCTGGGTACC ACTATGGGGT | |
| AGCATCATGT GAAGCCTGCA AGGCATTCTT CAAGAGGACA ATTCAAGGCA | |
| ATATAGAATA CAGCTGCCCT GCCACGAATG AATGTGAAAT CACAAAGCGC | |
| AGACGTAAAT CCTGCCAGGC TTGCCGCTTC ATGAAGTGTT TAAAAGTGGG | |
| CATGCTGAAA GAAGGGGTGC GTCTTGACAG AGTACGTGGA GGTCGGCAGA | |
| AGTACAAGCG CAGGATAGAT GCGGAGAACA GCCCATACCT GAACCCTCAG | |
| CTGGTTCAGC CAGCCAAAAA GCCATATAAC AAGATTGTCT CACATTTGTT | |
| GGTGGCTGAA CCGGAGAAGA TCTATGCCAT GCCTGACCCT ACTGTCCCCG | |
| ACAGTGACAT CAAAGCCCTC ACTACACTGT GTGACTTGGC CGACCGAGAG | |
| TTGGTGGTTA TCATTGGATG GGCGAAGCAT ATTCCAGGCT TCTCCACGCT | |
| GTCCCTGGCG GACCAGATGA GCCTTCTGCA GAGTGCTTGG ATGGAAATTT | |
| TGATCCTTGG TGTCGTATAC CGGTCTCTTT CATTTGAGGA TGAACTTGTC | |
| TATGCAGACG ATTATATAAT GGACGAAGAC CAGTCCAAAT TAGCAGGCCT | |
| TCTTGATCTA AATAATGCTA TCCTGCAGCT GGTAAAGAAA TACAAGAGCA | |
| TGAAGCTGGA AAAAGAAGAA TTTGTCACCC TCAAAGCTAT AGCTCTTGCT | |
| AATTCAGACT CCATGCACAT AGAAGATGTT GAAGCCGTTC AGAAGCTTCA | |
| GGATGTCTTA CATGAAGCGC TGCAGGATTA TGAAGCTGGC CAGCACATGG | |
| AAGACCCTCG TCGAGCTGGC AAGATGCTGA TGACACTGCC ACTCCTGAGG | |
| CAGACCTCTA CCAAGGCCGT GCAGCATTTC TACAACATCA AACTAGAAGG | |
| CAAAGTCCCA ATGCACAAAC TTTTTTTGGA AATGTTGGAG GCCAAGGTCT | |
| GACTAAAAGC TCCCTGGGCC TTCCCATCCT TCATGTTGAA AAAGGGAAAA | |
| TAAACCCAAG AGTGATGTCG AAGAAACTTA GAGTTTAGTT AACAACATCA | |
| AAAATCAACA GACTGCACTG ATAATTTAGC AGCAAGACTA TGAAGCAGCT | |
| TTCAGATTCC TCCATAGGTT CCTGATGAGT TCTTTCTACT TTCTCCATCA | |
| TCTTCTTTCC TCTTTCTTCC CACATTTCTC TTTCTCTTTA TTTTTTCTCC | |
| TTTTCTTCTT TCACCTCCCT TATTTCTTTG CTTCTTTCAT TCCTAGTTCC | |
| CATTCTCCTT TATTTTCTTC CCGTCTGCCT GCCTTCTTTC TTTTCTTTAC | |
| CTACTCTCAT TCCTCTCTTT TCTCATCCTT CCCCTTTTTT CTAAATTTGA | |
| AATAGCTTTA GTTTAAAAAA AAAAATCCTC CCTTCCCCCT TTCCTTTCCC | |
| TTTCTTTCCT TTTTCCCTTT CCTTTTCCCT TTCCTTTCCT TTCCTCTTGA | |
| CCTTCTTTCC ATCTTTCTTT TTCTTCCTTC TGCTGCTGAA CTTTTAAAAG | |
| AGGTCTCTAA CTGAAGAGAG ATGGAAGCCA GCCCTGCCAA AGGATGGAGA | |
| TCCATAATAT GGATGCCAGT GAACTTATTG TGAACCATAC CGTCCCCAAT | |
| GACTAAGGAA TCAAAGAGAG AGAACCAACG TTCCTAAAAG TACAGTGCAA | |
| CATATACAAA TTGACTGAGT GCAGTATTAG ATTTCATGGG AGCAGCCTCT | |
| AATTAGACAA CTTAAGCAAC GTTGCATCGG CTGCTTCTTA TCATTGCTTT | |
| TCCATCTAGA TCAGTTACAG CCATTTGATT CCTTAATTGT TTTTTCAAGT | |
| CTTCCAGGTA TTTGTTAGTT TAGCTACTAT GTAACTTTTT CAGGGAATAG | |
| TTTAAGCTTT ATTCATTCAT GCAATACTAA AGAGAAATAA GAATACTGCA | |
| ATTTTGTGCT GGCTTTGAAC AATTACGAAC AATAATGAAG GACAAATGAA | |
| TCCTGAAGGA AGATTTTTAA AAATGTTTTG TTTCTTCTTA CAAATGGAGA | |
| TTTTTTTGTA CCAGCTTTAC CACTTTTCAG CCATTTATTA ATATGGGAAT | |
| TTAACTTACT CAAGCAATAG TTGAAGGGAA GGTGCATATT ATCACGGATG | |
| CAATTTATGT TGTGTGCCAG TCTGGTCCCA AACATCAATT TCTTAACATG | |
| AGCTCCAGTT TACCTAAATG TTCACTGACA CAAAGGATGA GATTACACCT | |
| ACAGTGACTC TGAGTAGTCA CATATATAAG CACTGCACAT GAGATATAGA | |
| TCCGTAGAAT TGTCAGGAGT GCACCTCTCT ACTTGGGAGG TACAATTGCC | |
| ATATGATTTC TAGCTGCCAT GGTGGTTAGG AATGTGATAC TGCCTGTTTG | |
| CAAAGTCACA GACCTTGCCT CAGAAGGAGC TGTGAGCCAG TATTCATTTA | |
| AGAGAATTCC ACCACACTGG CGGCCCGCGC TTGAT. | |

The present invention also relates to an isolated and purified DNA molecule which encodes a truncated version of nNR2 referred to as nNR2-1. This cDNA molecule is set forth in SEQ ID NO:5 and is disclosed as follows:

| | |
|---|---|
| GCGGGCCGCC AGTGTGGTGG AATTCGGCTT GTCACTAGGA GAACATTTGT | (SEQ ID NO:5) |
| GTTAATTGCA CTGTGCTCTG TCAAGGAAAC TTTGATTTAT AGCTGGGGTG | |
| CACAAATAAT GGTTGCCGGT CGCACATGGA TTCGGTAGAA CTTTGCCTTC | |
| CTGAATCTTT TTCCCTGCAC TACGAGGAAG AGCTTCTCTG CAGAATGTCA | |
| AACAAAGATC GACACATTGA TTCCAGCTGT TCGTCCTTCA TCAAGACGGA | |
| ACCTTCCAGC CCAGCCTCCC TGACGGACAG CGTCAACCAC CACAGCCCTG | |
| GTGGCTCTTC AGACGCCAGT GGGAGCTACA GTTCAACCAT GAATGGCCAT | |
| CAGAACGGAC TTGACTCGCC ACCTCTCTAC CCTTCTGCTC CTATCCTGGG | |
| AGGTAGTGGG CCTGTCAGGA AACTGTATGA TGACTGCTCC AGCACCATTG | |
| TTGAAGATCC CCAGACCAAG TGTGAATACA TGCTCAACTC GATGCCCAAG | |
| AGACTGTGTT TAGTGTGTGG TGACATCGCT TCTGGGTACC ACTATGGGGT | |
| AGCATCATGT GAAGCCTGCA AGGCATTCTT CAAGAGGACA ATTCAAGGCA | |
| ATATAGAATA CAGCTGCCCT GCCACGAATG AATGTGAAAT CACAAAGCGC | |
| AGACGTAAAT CCTGCCAGGC TTGCCGCTTC ATGAAGTGTT TAAAAGTGGG | |
| CATGCTGAAA GAAGGGGTGC GTCTTGACAG AGTACGTGGA GGTCGGCAGA | |
| AGTACAAGCG CAGGATAGAT GCGGAGAACA GCCCATACCT GAACCCTCAG | |
| CTGGTTCAGC CAGCCAAAAA GCCATATAAC AAGATTGTCT CACATTTGTT | |
| GGTGGCTGAA CCGGAGAAGA TCTATGCCAT GCCTGACCCT ACTGTCCCCG | |
| ACAGTGACAT CAAAGCCCTC ACTACACTGT GTGACTTGGC CGACCGAGAG | |
| TTGGTGGTTA TCATTGGATG GGCGAAGCAT ATTCCAGGCT TCTCCACGCT | |
| GTCCCTGGCG GACCAGATGA GCCTTCTGCA GAGTGCTTGG ATGGAAATTT | |
| TGATCCTTGG TGTCGTATAC CGGTCTCTTT CATTTGAGGA TGAACTTGTC | |
| TATGCAGACG ATTATATAAT GGACGAAGAC CAGTCCAAAT TAGCAGGCCT | |
| TCTTGATCTA AATAATGCTA TCCTGCAGCT GGTAAAGAAA TACAAGAGCA | |
| TGAAGCTGGA AAAAGAAGAA TTTGTCACCC TCAAAGCTAT AGCTCTTGCT | |
| AATTCAGACT CCATGCACAT AGAAGATGTT GAAGCCGTTC AGAAGCTTCA | |
| GGATGTCTTA CATGAAGCGC TGCAGGATTA TGAAGCTGGC CAGCACATGG | |
| AGAAGACCCT CGTCGAGCTG GCAAGATGCT GATGACACTG CCACTCCTGA | |
| GGCAGACCTC TACCAAGGCC GTGCAGCATT TCTACAACAT CAAACTAGAA | |
| GGCAAAGTCC CAATGCACAA ACTTTTTTTG GAAATGTTGG AGGCCAAGGT | |
| CTGACTAAAA GCTCCCTGGG CCTTCCCATC CTTCATGTTG AAAAAGGGAA | |
| AATAAACCCA AGAGTGATGT CGAAGAAACT TAGAGTTTAG TTAACAACAT | |
| CAAAAATCAA CAGACTGCAC TGATAATTTA GCAGCAAGAC TATGAAGCAG | |
| CTTTCAGATT CCTCCATAGG TTCCTGATGA GTTCTTTCTA CTTTCTCCAT | |
| CATCTTCTTT CCTCTTTCTT CCCACATTTC TCTTTCTCTT TATTTTTTCT | |
| CCTTTTCTTC TTTCACCTCC CTTATTTCTT TGCTTCTTTC ATTCCTAGTT | |
| CCCATTCTCC TTTATTTTCT TCCCGTCTGC CTGCCTTCTT TCTTTTCTTT | |
| ACCTACTCTC ATTCCTCTCT TTTCTCATCC TTCCCCTTTT TTCTAAATTT | |
| GAAATAGCTT TAGTTTAAAA AAAAAAATCC TCCCTTCCCC CTTTCCTTTC | |
| CCTTTCTTTC CTTTTTCCCT TTCCTTTTCC CTTTCCTTTC CTTTCCTCTT | |
| GACCTTCTTT CCATCTTTCT TTTTCTTCCT TCTGCTGCTG AACTTTTAAA | |
| AGAGGTCTCT AACTGAAGAG AGATGGAAGC CAGCCCTGCC AAAGGATGGA | |
| GATCCATAAT ATGGATGCCA GTGAACTTAT TGTGAACCAT ACCGTCCCCA | |
| ATGACTAAGG AATCAAAGAG AGAGAACCAA CGTTCCTAAA AGTACAGTGC | |
| AACATATACA AATTGACTGA GTGCAGTATT AGATTTCATG GGAGCAGCCT | |
| CTAATTAGAC AACTTAAGCA ACGTTGCATC GGCTGCTTCT TATCATTGCT | |
| TTTCCATCTA GATCAGTTAC AGCCATTTGA TTCCTTAATT GTTTTTTCAA | |
| GTCTTCCAGG TATTTGTTAG TTTAGCTACT ATGTAACTTT TTCAGGGAAT | |
| AGTTTAAGCT TTATTCATTC ATGCAATACT AAAGAGAAAT AAGAATACTG | |
| CAATTTTGTG CTGGCTTTGA ACAATTACGA ACAATAATGA AGGACAAATG | |
| AATCCTGAAG GAAGATTTTT AAAAATGTTT TGTTTCTTCT TACAAATGGA | |
| GATTTTTTTG TACCAGCTTT ACCACTTTTC AGCCATTTAT TAATATGGGA | |
| ATTTAACTTA CTCAAGCAAT AGTTGAAGGG AAGGTGCATA TTATCACGGA | |
| TGCAATTTAT GTTGTGTGCC AGTCTGGTCC CAAACATCAA TTTCTTAACA | |
| TGAGCTCCAG TTTACCTAAA TGTTCACTGA CACAAAGGAT GAGATTACAC | |
| CTACAGTGAC TCTGAGTAGT CACATATATA AGCACTGCAC ATGAGATATA | |
| GATCCGTAGA ATTGTCAGGA GTGCACCTCT CTACTTGGGA GGTACAATTG | |
| CCATATGATT TCTAGCTGCC ATGGTGGTTA GGAATGTGAT ACTGCCTGTT | |
| TGCAAAGTCA CAGACCTTGC CTCAGAAGGA GCTGTGAGCC AGTATTCATT | |
| TAAGAGAATT CCACCACACT GGCGGCCCGC GCTTGAT | |

The present invention also relates to a substantially purified form of the novel nuclear trans-acting receptor protein, nNR1, which is shown in FIGS. 2A-F and FIG. 3 and as set forth in SEQ ID NO:2, disclosed as follows:

| | |
|---|---|
| MSSDDRHLGS SCGSFIKTEP SSPSSGIDAL SHHSPSGSSD ASGGFGLALG | (SEQ ID NO:2) |
| THANGLDSPP MFAGAGLGGT PCRKSYEDCA SGIMEDSAIK CEYMLNAIPK | |
| RLCLVCGDIA SGYHYGVASC EACKAFFKRT IQGNIEYSCP ATNECEITKR | |
| RRKSCQACRF MKCLKVGMLK EGVRLDRVRG GRQKYKRRLD SESSPYLSLQ | |
| ISPPAKKPLT KIVSYLLVAE PDKLYAMPPP GMPEGDIKAL TTLCDLADRE | |
| LVVIIGWAKH IPGFSSLSLG DQMSLLQSAW MEILILGIVY RSLPYDDKLV | |
| YAEDYIMDEE HSRLAGLLEL YRAILQLVRR YKKLKVEKEE FVTLKALALA | |
| NSDSMYIEDL EAVQKLQDLL HEALQDYELS QRHEEPWRTG KLLLTLPLLR | |
| QTAAKAVQHF YSVKLQGKVP MHKLFLEMLE AKAWARADSL QEWRPLEQVP | |
| SPLHPATKRQ HVHFLTPLPP PPSVAWVGTA QAGYHLEVFL PQRAGWPRAA. | |

The present invention also relates to biologically active fragments and/or mutants of nNR1 as set forth as SEQ ID NO:2, including but not necessarily limited to amino acid substitutions, deletions, additions, amino terminal truncations and carboxy-terminal truncations such that these mutations provide for proteins or protein fragments of diagnostic, therapeutic or prophylactic use and would be useful for screening for agonists and/or antagonists of nNR1 function.

The present invention also relates to a substantially purified form of the novel nuclear trans-acting receptor protein, nNR2, which is shown in FIGS. 5A-E and FIG. 6 and as set forth in SEQ ID NO:4, disclosed as follows:

| | |
|---|---|
| MDSVELCLPE SFSLHYEEEL LCRMSNKDRH IDSSCSSFIK TEPSSPASLT | (SEQ ID NO:4) |
| DSVNHHSPGG SSDASGSYSS TMNGHQNGLD SPPLYPSAPI LGGSGPVRKL | |
| YDDCSSTIVE DPQTKCEYML NSMPKRLCLV CGDIASGYHY GVASCEACKA | |
| FFKRTIQGNI EYSCPATNEC EITKRRRKSC QACRFMKCLK VGMLKEGVRL | |
| DRVRGGRQKY KRRIDAENSP YLNPQLVQPA KKPYNKIVSH LLVAEPEKIY | |
| AMPDPTVPDS DIKALTTLCD LADRELVVII GWAKHIPGFS TLSLADQMSL | |
| LQSAWMEILI LGVVYRSLSF EDELVYADDY IMDEDQSKLA GLLDLNNAIL | |
| QLVKKYKSMK LEKEEFVTLK AIALANSDSM HIEDVEAVQK LQDVLHEALQ | |
| DYEAGQHMED PRRAGKMLMT LPLLRQTSTK AVQHFYNIKL EGKVPMHKLF | |
| LEMLEAKV. | |

The present invention also relates to biologically active fragments and/or mutants of nNR2 as set forth as SEQ ID NO:4, including but not necessarily limited to amino acid substitutions, deletions, additions, amino terminal truncations and carboxy-terminal truncations such that these mutations provide for proteins or protein fragments of diagnostic, therapeutic or prophylactic use and would be useful for screening for agonists and/or antagonists of nNR2 function. To this end, an example of such a protein is the carboxy-terminal truncated version of nNR2, referred to as nNR2-1 and described in FIG. 8 and set forth as SEQ ID NO:6, as follows:

| | |
|---|---|
| MDSVELCLPE SFSLHYEEEL LCRMSNKDRH IDSSCSSFIK TEPSSPASLT | (SEQ ID NO:6) |
| DSVNHHSPGG SSDASGSYSS TMNGHQNGLD SPPLYPSAPI LGGSGPVRKL | |
| YDDCSSTIVE DPQTKCEYML NSMPKRLCLV CGDIASGYHY GVASCEACKA | |
| FFKRTIQGNI EYSCPATNEC EITKRRRKSC QACRFMKCLK VGMLKEGVRL | |
| DRVRGGRQKY KRRIDAENSP YLNPQLVQPA KKPYNKIVSH LLVAEPEKIY | |
| AMPDPTVPDS DIKALTTLCD LADRELVVII GWAKHIPGFS TLSLADQMSL | |
| LQSAWMEILI LGVVYRSLSF EDELVYADDY IMDEDQSKLA GLLDLNNAIL | |
| QLVKKYKSMK LEKEEFVTLK AIALANSDSM HIEDVEAVQK LQDVLHEALQ | |
| DYEAGQHMEK TLVELARC. | |

The present invention also relates to isolated nucleic acid molecules which are fusion constructions expressing fusion proteins useful in assays to identify compounds which modulate wild-type human nNR1, nNR2 and/or nNR2-1 activity. A preferred aspect of this portion of the invention includes, but is not limited to, glutathione S-transferase GST-nNR1 and/or GST-nNR2 fusion constructs. These fusion constructs include, but are not limited to, all or a portion of the ligand-binding domain of nNR1, nNR2 and/or nNR2-1, respectively, as an in-frame fusion at the carboxy terminus of the GST gene. The disclosure of SEQ ID NOS:1-4 allow the artisan of ordinary skill to construct any such nucleic acid molecule encoding a GST-nuclear receptor fusion protein. Soluble recombinant GST-nuclear receptor fusion proteins may be expressed in various expression systems, including Spodoptera frugiperda (Sf21) insect cells (Invitrogen) using a baculovirus expression vector (e.g., Bac-N-Blue DNA from Invitrogen or pAcG2T from Pharmingen).

The isolated nucleic acid molecule of the present invention may include a deoxyribonucleic acid molecule (DNA), such as genomic DNA and complementary DNA (cDNA), which may be single (coding or noncoding strand) or double stranded, as well as synthetic DNA, such as a synthesized, single stranded polynucleotide. The isolated nucleic acid molecule of the present invention may also include a ribonucleic acid molecule (RNA).

It is known that there is a substantial amount of redundancy in the various codons which code for specific amino acids. Therefore, this invention is also directed to those DNA sequences encode RNA comprising alternative codons which code for the eventual translation of the identical amino acid, as shown below:

A=Ala=Alanine: codons GCA, GCC, GCG, GCU

C=Cys=Cysteine: codons UGC, UGU

D=Asp=Aspartic acid: codons GAC, GAU

E=Glu=Glutamic acid: codons GAA, GAG

F=Phe=Phenylalanine: codons UUC, UUU

G=Gly=Glycine: codons GGA, GGC, GGG, GGU

H=His=Histidine: codons CAC, CAU

I=Ile=Isoleucine: codons AUA, AUC, AUU

K=Lys=Lysine: codons AAA, AAG

L=Leu=Leucine: codons UUA, UUG, CUA, CUC, CUG, CUU

M=Met=Methionine: codon AUG

N=Asp=Asparagine: codons AAC, AAU

P=Pro=Proline: codons CCA, CCC, CCG, CCU

Q=Gln=Glutamine: codons CAA, CAG

R=Arg=Arginine: codons AGA, AGG, CGA, CGC, CGG, CGU

S=Ser=Serine: codons AGC, AGU, UCA, UCC, UCG, UCU

T=Thr=Threonine: codons ACA, ACC, ACG, ACU

V=Val=Valine: codons GUA, GUC, GUG, GUU

W=Trp=Tryptophan: codon UGG

Y=Tyr=Tyrosine: codons UAC, UAU

Therefore, the present invention discloses codon redundancy which may result in differing DNA molecules expressing an identical protein. For purposes of this specification, a sequence bearing one or more replaced codons will be defined as a degenerate variation. Also included within the scope of this invention are mutations either in the DNA sequence or the translated protein which do not substantially alter the ultimate physical properties of the expressed protein. For example, substitution of valine for leucine, arginine for lysine, or asparagine for glutamine may not cause a change in functionality of the polypeptide.

It is known that DNA sequences coding for a peptide may be altered so as to code for a peptide having properties that are different than those of the naturally occurring peptide. Methods of altering the DNA sequences include but are not limited to site directed mutagenesis. Examples of altered properties include but are not limited to changes in the affinity of an enzyme for a substrate or a receptor for a ligand.

As used herein, “purified” and “isolated” are utilized interchangeably to stand for the proposition that the nucleic acid, protein, or respective fragment thereof in question has been substantially removed from its in vivo environment so that it may be manipulated by the skilled artisan, such as but not limited to nucleotide sequencing, restriction digestion, site-directed mutagenesis, and subcloning into expression vectors for a nucleic acid fragment as well as obtaining the protein or protein fragment in pure quantities so as to afford the opportunity to generate polyclonal antibodies, monoclonal antibodies, amino acid sequencing, and peptide digestion. Therefore, the nucleic acids claimed herein may be present in whole cells or in cell lysates or in a partially purified or substantially purified form. A nucleic acid is considered substantially purified when it is purified away from environmental contaminants. Thus, a nucleic acid sequence isolated from cells is considered to be substantially purified when purified from cellular components by standard methods while a chemically synthesized nucleic acid sequence is considered to be substantially purified when purified from its chemical precursors.

The present invention also relates to recombinant vectors and recombinant hosts, both prokaryotic and eukaryotic, which contain the substantially purified nucleic acid molecules disclosed throughout this specification.

Therefore, the present invention also relates to methods of expressing nNR1, nNR2 and/or nNR2-1 and biological equivalents disclosed herein, assays employing these recombinantly expressed gene products, cells expressing these gene products, and agonistic and/or antagonistic compounds identified through the use of assays utilizing these recombinant forms, including, but not limited to, one or more modulators of the human nNR1, nNR2 and/or nNR2-1 either through direct contact LBD or through direct or indirect contact with a ligand which either interacts with the DBD or with the wild-type transcription complex which either nNR1, nNR2 and/or nNR2-1 interacts in trans, thereby modulating cell differentiation or cell development.

As used herein, a “biologically active equivalent” or “functional derivative” of a wild-type human nNR1, nNR2 and/or nNR2-1 possesses a biological activity that is substantially similar to the biological activity of the wild type human nNR1, nNR2 and/or nNR2-1. The term “functional derivative” is intended to include the “fragments,” “mutants,” “variants,” “degenerate variants,” “analogs” and “homologues” or to “chemical derivatives” of the wild type human nNR1, nNR2 and/or nNR2-1 protein. The term “fragment” is meant to refer to any polypeptide subset of wild-type human nNR1 or nNR2. The term “mutant” is meant to refer to a molecule that may be substantially similar to the wild-type form but possesses distinguishing biological characteristics. Such altered characteristics include but are in no way limited to altered substrate binding, altered substrate affinity and altered sensitivity to chemical compounds affecting biological activity of the human nNR1, nNR2 and/or nNR2-1 or human nNR1, nNR2 and/or nNR2-1 functional derivatives. The term “variant” is meant to refer to a molecule substantially similar in structure and function to either the entire wild-type protein or to a fragment thereof. A molecule is “substantially similar” to a wild-type human nNR1, nNR2 and/or nNR2-1-like protein if both molecules have substantially similar structures or if both molecules possess similar biological activity. Therefore, if the two molecules possess substantially similar activity, they are considered to be variants even if the structure of one of the molecules is not found in the other or even if the two amino acid sequences are not identical. The term “analog” refers to a molecule substantially similar in function to either the full-length human nNR1, nNR2 and/or nNR2-1 protein or to a biologically active fragment thereof.

Any of a variety of procedures may be used to clone human nNR1, nNR2 and/or nNR2-1. These methods include, but are not limited to, (1) a RACE PCR cloning technique (Frohman, et al., 1988, Proc. Natl. Acad. Sci. USA 85: 8998-9002). 5′ and/or 3′ RACE may be performed to generate a full-length cDNA sequence. This strategy involves using gene-specific oligonucleotide primers for PCR amplification of human nNR1, nNR2 and/or nNR2-1 cDNA. These gene-specific primers are designed through identification of an expressed sequence tag (EST) nucleotide sequence which has been identified by searching any number of publicly available nucleic acid and protein databases; (2) direct functional expression of the human nNR1, nNR2 and/or nNR2-1 cDNA following the construction of a human nNR1, nNR2 and/or nNR2-1-containing cDNA library in an appropriate expression vector system; (3) screening a human nNR1, nNR2 and/or nNR2-1-containing cDNA library constructed in a bacteriophage or plasmid shuttle vector with a labeled degenerate oligonucleotide probe designed from the amino acid sequence of the human nNR1, nNR2 and/or nNR2-1 protein; (4) screening a human nNR1, nNR2 and/or nNR2-1-containing cDNA library constructed in a bacteriophage or plasmid shuttle vector with a partial cDNA encoding the human nNR1, nNR2 and/or nNR2-1 protein. This partial cDNA is obtained by the specific PCR amplification of human nNR1, nNR2 and/or nNR2-1 DNA fragments through the design of degenerate oligonucleotide primers from the amino acid sequence known for other kinases which are related to the human nNR1, nNR2 and/or nNR2-1 protein; (5) screening a human nNR1, nNR2 and/or nNR2-1-containing cDNA library constructed in a bacteriophage or plasmid shuttle vector with a partial cDNA encoding the human nNR1, nNR2 and/or nNR2-1 protein. This strategy may also involve using gene-specific oligonucleotide primers for PCR amplification of human nNR1, nNR2 and/or nNR2-1 cDNA identified as an EST as described above; or (6) designing 5′ and 3′ gene specific oligonucleotides using SEQ ID NO: 1 as a template so that either the full-length cDNA may be generated by known PCR techniques, or a portion of the coding region may be generated by these same known PCR techniques to generate and isolate a portion of the coding region to use as a probe to screen one of numerous types of cDNA and/or genomic libraries in order to isolate a full-length version of the nucleotide sequence encoding human nNR1, nNR2 and/or nNR2-1.

It is readily apparent to those skilled in the art that other types of libraries, as well as libraries constructed from other cell types-or species types, may be useful for isolating a nNR1, nNR2 and/or nNR2-1-encoding DNA or a nNR1, nNR2 and/or nNR2-1 homologue. Other types of libraries include, but are not limited to, cDNA libraries derived from other cells or cell lines other than human cells or tissue such as murine cells, rodent cells or any other such vertebrate host which may contain nNR1, nNR2 and/or nNR2-1-encoding DNA. Additionally a nNR1, nNR2 and/or nNR2-1 gene and homologues may be isolated by oligonucleotide- or polynucleotide-based hybridization screening of a vertebrate genomic library, including but not limited to, a murine genomic library, a rodent genomic library, as well as concomitant human genomic DNA libraries.

It is readily apparent to those skilled in the art that suitable cDNA libraries may be prepared from cells or cell lines which have nNR1, nNR2 and/or nNR2-1 activity. The selection of cells or cell lines for use in preparing a cDNA library to isolate a cDNA encoding nNR1, nNR2 and/or nNR2-1 may be done by first measuring cell-associated nNR1, nNR2 and/or nNR2-1 activity using any known assay available for such a purpose.

Preparation of cDNA libraries can be performed by standard techniques well known in the art. Well known cDNA library construction techniques can be found for example, in Sambrook et al., 1989, Molecular Cloning: A Laboratory Manual; Cold Spring Harbor Laboratory, Cold Spring Harbor, N.Y. Complementary DNA libraries may also be obtained from numerous commercial sources, including but not limited to Clontech Laboratories, Inc. and Stratagene.

It is also readily apparent to those skilled in the art that DNA encoding human nNR1, nNR2 and/or nNR2-1 may also be isolated from a suitable genomic DNA library. Construction of genomic DNA libraries can be performed by standard techniques well known in the art. Well known genomic DNA library construction techniques can be found in Sambrook, et al., supra.

In order to clone the human nNR1, nNR2 and/or nNR2-1 gene by one of the preferred methods, the amino acid sequence or DNA sequence of human nNR1, nNR2 and/or nNR2-1 or a homologous protein may be necessary. To accomplish this, the nNR1, nNR2 and/or nNR2-1 protein or a homologous protein may be purified and partial amino acid sequence determined by automated sequenators. It is not necessary to determine the entire amino acid sequence, but the linear sequence of two regions of 6 to 8 amino acids can be determined for the PCR amplification of a partial human nNR1, nNR2 and/or nNR2-1 DNA fragment. Once suitable amino acid sequences have been identified, the DNA sequences capable of encoding them are synthesized. Because the genetic code is degenerate, more than one codon may be used to encode a particular amino acid, and therefore, the amino acid sequence can be encoded by any of a set of similar DNA oligonucleotides. Only one member of the set will be identical to the human nNR1, nNR2 and/or nNR2-1 sequence but others in the set will be capable of hybridizing to human nNR1, nNR2 and/or nNR2-1 DNA even in the presence of DNA oligonucleotides with mismatches. The mismatched DNA oligonucleotides may still sufficiently hybridize to the human nNR1, nNR2 and/or nNR2-1 DNA to permit identification and isolation of human nNR1, nNR2 and/or nNR2-1 encoding DNA. Alternatively, the nucleotide sequence of a region of an expressed sequence may be identified by searching one or more available genomic databases. Gene-specific primers may be used to perform PCR amplification of a cDNA of interest from either a cDNA library or a population of cDNAs. As noted above, the appropriate nucleotide sequence for use in a PCR-based method may be obtained from SEQ ID NO: 1, either for the purpose of isolating overlapping 5′ and 3′ RACE products for generation of a full-length sequence coding for human nNR1, nNR2 and/or nNR2-1, or to isolate a portion of the nucleotide sequence coding for human nNR1, nNR2 and/or nNR2-1 for use as a probe to screen one or more cDNA- or genomic-based libraries to isolate a full-length sequence encoding human nNR1, nNR2 and/or nNR2-1 or human nNR1, nNR2 and/or nNR2-1-like proteins.

In an exemplified method, the human nNR1, nNR2 and/or nNR2-1 full-length cDNA of the present invention were generated by PCR scanning human cDNA libraries with oligonucleotide primers generated from ESTs showing homology to hERR2. Briefly, random and oligo dT primed cDNA libraries as described herein which consist of approximately 4 million primary clones were constructed in the plasmid vector pBluescript (Stratagene, LaJolla, Calif.). The primary clones were subdivided into 188 pools with each pool containing ˜20,000 clones. Each pool was amplified separately and the resulting plasmid pools were collected and transferred into two 96-well plates. Primer pairs from the 5′ and 3′ portion of an EST are used to scan the respective cDNA library distributed in a 96-well plate. Initial positive pools are identified with EST primers. Corresponding full length cDNA clones were retrieved via inverse PCR using primer pairs designed from the EST which are back to back against each other. Therefore, the primers walk away from each other during the PCR reaction, resulting in amplification of a population of linearized plasmid DNA molecules corresponding to the EST. cDNA clones were obtained by ligating linear DNA and transforming the circularized DNA into bacteria competent cells. Usually, four positive clones for each gene were used for sequence analysis because of the possibility of mutation during long PCR reactions. The consensus DNA sequence is considered as the wild type DNA sequence. Recloning of the gene through PCR using gene specific primers covering the whole open reading frame was done so as to obtain a cDNA clone which has an identical DNA sequence to the consensus sequence. This procedure does not depend upon using a cDNA library with directionally cloned inserts, but does require cDNA libraries constructed in a plasmid vector, such as pBluescript. This procedure was utilized to identify full length cDNA molecules representing human nNR1, nNR2 and/or nNR2-1.

A variety of mammalian expression vectors may be used to express recombinant human nNR1, nNR2 and/or nNR2-1 in mammalian cells. Expression vectors are defined herein as DNA sequences that are required for the transcription of cloned DNA and the translation of their mRNAs in an appropriate host. Such vectors can be used to express eukaryotic DNA in a variety of hosts such as bacteria, blue green algae, plant cells, insect cells and animal cells. Specifically designed vectors allow the shuttling of DNA between hosts such as bacteria-yeast or bacteria-animal cells. An appropriately constructed expression vector should contain: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one which causes mRNAs to be initiated at high frequency. Expression vectors may include, but are not limited to, cloning vectors, modified cloning vectors, specifically designed plasmids or viruses.

Commercially available mammalian expression vectors which may be suitable for recombinant human nNR1, nNR2 and/or nNR2-1 expression, include but are not limited to, pcDNA3.1 (Invitrogen), pLITMUS28, pLITMUS29, pLITMUS38 and pLITMUS39 (New England Bioloabs), pcDNAI, pcDNAIamp (Invitrogen), pcDNA3 (Invitrogen), pMC1neo (Stratagene), pXT1 (Stratagene), pSG5 (Stratagene), EBO-pSV2-neo (ATCC 37593) pBPV-1(8-2) (ATCC 37110), pdBPV-Tneo(342-12) (ATCC 37224), pRSVgpt (ATCC 37199), pRSVneo (ATCC 37198), pSV2-dhfr (ATCC 37146), pUCTag (ATCC 37460), and 1ZD35 (ATCC 37565).

A variety of bacterial expression vectors may be used to express recombinant human nNR1, nNR2 and/or nNR2-1 in bacterial cells. Commercially available bacterial expression vectors which may be suitable for recombinant human nNR1, nNR2 and/or nNR2-1 expression include, but are not limited to pQE (Qiagen), pET11a (Novagen), lambda gt11 (Invitrogen), and pKK223-3 (Pharmacia).

A variety of fungal cell expression vectors may be used to express recombinant human nNR1, nNR2 and/or nNR2-1 in fungal cells. Commercially available fungal cell expression vectors which may be suitable for recombinant human nNR1, nNR2 and/or nNR2-1 expression include but are not limited to pYES2 (Invitrogen) and Pichia expression vector (Invitrogen).

A variety of insect cell expression vectors may be used to express recombinant receptor in insect cells. Commercially available insect cell expression vectors which may be suitable for recombinant expression of human nNR1, nNR2 and/or nNR2-1 include but are not limited to pBlueBacIII and pBlueBacHis2 (Invitrogen), and pAcG2T (Pharmingen).

An expression vector containing DNA encoding a human nNR1, nNR2 and/or nNR2-1-like protein may be used for expression of human nNR1, nNR2 and/or nNR2-1 in a recombinant host cell. Recombinant host cells may be prokaryotic or eukaryotic, including but not limited to bacteria such as E. coli, fungal cells such as yeast, mammalian cells including but not limited to cell lines of human, bovine, porcine, monkey and rodent origin, and insect cells including but not limited to Drosophila- and silkworm-derived cell lines. Cell lines derived from mammalian species which may be suitable and which are commercially available, include but are not limited to, L cells L-M(TK⁻) (ATCC CCL 1.3), L cells L-M (ATCC CCL 1.2), Saos-2 (ATCC HTB-85), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26), MRC-5 (ATCC CCL 171) and CPAE (ATCC CCL 209).

The expression vector may be introduced into host cells via any one of a number of techniques including but not limited to transformation, transfection, protoplast fusion, and electroporation. The expression vector-containing cells are individually analyzed to determine whether they produce human nNR1, nNR2 and/or nNR2-1 protein. Identification of human nNR1, nNR2 and/or nNR2-1 expressing cells may be done by several means, including but not limited to immunological reactivity with anti-human nNR1, nNR2 and/or nNR2-1 antibodies, labeled ligand binding and the presence of host cell-associated human nNR1, nNR2 and/or nNR2-1 activity.

The cloned human nNR1, nNR2 and/or nNR2-1 cDNA obtained through the methods described above may be recombinantly expressed by molecular cloning into an expression vector (such as pcDNA3.1, pQE, pBlueBacHis2 and pLITMUS28) containing a suitable promoter and other appropriate transcription regulatory elements, and transferred into prokaryotic or eukaryotic host cells to produce recombinant human nNR1, nNR2 and/or nNR2-1. Techniques for such manipulations can be found described in Sambrook, et al., supra, are discussed at length in the Example section and are well known and easily available to the artisan of ordinary skill in the art.

Expression of human nNR1, nNR2 and/or nNR2-1 DNA may also be performed using in vitro produced synthetic mRNA. Synthetic mRNA can be efficiently translated in various cell-free systems, including but not limited to wheat germ extracts and reticulocyte extracts, as well as efficiently translated in cell based systems, including but not limited to microinjection into frog oocytes, with microinjection into frog oocytes being preferred.

To determine the human nNR1, nNR2 and/or nNR2-1 cDNA sequence(s) that yields optimal levels of human nNR1, nNR2 and/or nNR2-1, cDNA molecules including but not limited to the following can be constructed: a cDNA fragment containing the full-length open reading frame for human nNR1, nNR2 and/or nNR2-1 as well as various constructs containing portions of the cDNA encoding only specific domains of the protein or rearranged domains of the protein. All constructs can be designed to contain none, all or portions of the 5′ and/or 3′ untranslated region of a human nNR1, nNR2 and/or nNR2-1 cDNA. The expression levels and activity of human nNR1, nNR2 and/or nNR2-1 can be determined following the introduction, both singly and in combination, of these constructs into appropriate host cells. Following determination of the human nNR1, nNR2 and/or nNR2-1 cDNA cassette yielding optimal expression in transient assays, this nNR1, nNR2 and/or nNR2-1 cDNA construct is transferred to a variety of expression vectors (including recombinant viruses), including but not limited to those for mammalian cells, plant cells, insect cells, oocytes, bacteria, and yeast cells.

The present invention also relates to polyclonal and monoclonal antibodies raised in response to either the human form of nNR1, nNR2 and/or nNR2-1 disclosed herein, or a biologically active fragment thereof It will be especially preferable to raise antibodies against epitopes within the NH₂-terminal domain of nNR1, nNR2 and/or nNR2-1, which show the least homology to other known proteins belonging to the human nuclear receptor superfamily.

Recombinant nNR1, nNR2 and/or nNR2-1 protein can be separated from other cellular proteins by use of an immunoaffinity column made with monoclonal or polyclonal antibodies specific for full-length nNR1, nNR2 and/or nNR2-1 protein, or polypeptide fragments of nNR1, nNR2 and/or nNR2-1 protein. Additionally, polyclonal or monoclonal antibodies may be raised against a synthetic peptide (usually from about 9 to about 25 amino acids in length) from a portion of the protein as disclosed in SEQ ID NO:2. Monospecific antibodies to human nNR1, nNR2 and/or nNR2-1 are purified from mammalian antisera containing antibodies reactive against human nNR1, nNR2 and/or nNR2-1 or are prepared as monoclonal antibodies reactive with human nNR1, nNR2 and/or nNR2-1 using the technique of Kohler and Milstein (1975, Nature 256: 495-497). Monospecific antibody as used herein is defined as a single antibody species or multiple antibody species with homogenous binding characteristics for human nNR1, nNR2 and/or nNR2-1. Homogenous binding as used herein refers to the ability of the antibody species to bind to a specific antigen or epitope, such as those associated with human nNR1, nNR2 and/or nNR2-1, as described above. Human nNR1, nNR2 and/or nNR2-1-specific antibodies are raised by immunizing animals such as mice, rats, guinea pigs, rabbits, goats, horses and the like, with an appropriate concentration of human nNR1, nNR2 and/or nNR2-1 protein or a synthetic peptide generated from a portion of human nNR1, nNR2 and/or nNR2-1 with or without an immune adjuvant.

Preimmune serum is collected prior to the first immunization. Each animal receives between about 0.1 mg and about 1000 mg of human nNR1, nNR2 and/or nNR2-1 protein associated with an acceptable immune adjuvant. Such acceptable adjuvants include, but are not limited to, Freund's complete, Freund's incomplete, alum-precipitate, water in oil emulsion containing Corynebacterium parvum and tRNA. The initial immunization consists of human nNR1, nNR2 and/or nNR2-1 protein or peptide fragment thereof in, preferably, Freund's complete adjuvant at multiple sites either subcutaneously (SC), intraperitoneally (IP) or both. Each animal is bled at regular intervals, preferably weekly, to determine antibody titer. The animals may or may not receive booster injections following the initial immunization. Those animals receiving booster injections are generally given an equal amount of human nNR1, nNR2 and/or nNR2-1 in Freund's incomplete adjuvant by the same route. Booster injections are given at about three week intervals until maximal titers are obtained. At about 7 days after each booster immunization or about weekly after a single immunization, the animals are bled, the serum collected, and aliquots are stored at about −20° C.

Monoclonal antibodies (mAb) reactive with human nNR1, nNR2 and/or nNR2-1 are prepared by immunizing inbred mice, preferably Balb/c, with human nNR1, nNR2 and/or nNR2-1 protein. The mice are immunized by the IP or SC route with about 1 mg to about 100 mg, preferably about 10 mg, of human nNR1, nNR2 and/or nNR2-1 protein in about 0.5 ml buffer or saline incorporated in an equal volume of an acceptable adjuvant, as discussed above. Freund's complete adjuvant is preferred. The mice receive an initial immunization on day 0 and are rested for about 3 to about 30 weeks. Immunized mice are given one or more booster immunizations of about 1 to about 100 mg of human nNR1, nNR2 and/or nNR2-1 in a buffer solution such as phosphate buffered saline by the intravenous (IV) route. Lymphocytes, from antibody positive mice, preferably splenic lymphocytes, are obtained by removing spleens from immunized mice by standard procedures known in the art. Hybridoma cells are produced by mixing the splenic lymphocytes with an appropriate fusion partner, preferably myeloma cells, under conditions which will allow the formation of stable hybridomas. Fusion partners may include, but are not limited to: mouse myelomas P3/NS1/Ag 4-1; MPC-11; S-194 and Sp 2/0, with Sp 2/0 being preferred. The antibody producing cells and myeloma cells are fused in polyethylene glycol, about 1000 mol. wt., at concentrations from about 30% to about 50%. Fused hybridoma cells are selected by growth in hypoxanthine, thymidine and aminopterin supplemented Dulbecco's Modified Eagles Medium (DMEM) by procedures known in the art. Supernatant fluids are collected form growth positive wells on about days 14, 18, and 21 and are screened for antibody production by an immunoassay such as solid phase immunoradioassay (SPIRA) using human nNR1, nNR2 and/or nNR2-1 as the antigen. The culture fluids are also tested in the Ouchterlony precipitation assay to determine the isotype of the mAb. Hybridoma cells from antibody positive wells are cloned by a technique such as the soft agar technique of MacPherson, 1973, Soft Agar Techniques, in Tissue Culture Methods and Applications, Kruse and Paterson, Eds., Academic Press.

Monoclonal antibodies are produced in vivo by injection of pristine primed Balb/c mice, approximately 0.5 ml per mouse, with about 2×10⁶ to about 6×10⁶ hybridoma cells about 4 days after priming. Ascites fluid is collected at approximately 8-12 days after cell transfer and the monoclonal antibodies are purified by techniques known in the art.

In vitro production of anti-human nNR1, nNR2 and/or nNR2-1 mAb is carried out by growing the hybridoma in DMEM containing about 2% fetal calf serum to obtain sufficient quantities of the specific mAb. The mAb are purified by techniques known in the art.

Antibody titers of ascites or hybridoma culture fluids are determined by various serological or immunological assays which include, but are not limited to, precipitation, passive agglutination, enzyme-linked immunosorbent antibody (ELISA) technique and radioimmunoassay (RIA) techniques. Similar assays are used to detect the presence of human nNR1, nNR2 and/or nNR2-1 in body fluids or tissue and cell extracts.

It is readily apparent to those skilled in the art that the above described methods for producing monospecific antibodies may be utilized to produce antibodies specific for human nNR1, nNR2 and/or nNR2-1 peptide fragments, or full-length human nNR1, nNR2 and/or nNR2-1.

Human nNR1, nNR2 and/or nNR2-1 antibody affinity columns are made, for example, by adding the antibodies to Affigel-10 (Biorad), a gel support which is pre-activated with N-hydroxysuccinimide esters such that the antibodies form covalent linkages with the agarose gel bead support. The antibodies are then coupled to the gel via amide bonds with the spacer arm. The remaining activated esters are then quenched with 1M ethanolamine HCl (pH 8). The column is washed with water followed by 0.23 M glycine HCl (pH 2.6) to remove any non-conjugated antibody or extraneous protein. The column is then equilibrated in phosphate buffered saline (pH 7.3) and the cell culture supernatants or cell extracts containing full-length human nNR1, nNR2 and/or nNR2-1 or human nNR1, nNR2 and/or nNR2-1 protein fragments are slowly passed through the column. The column is then washed with phosphate buffered saline until the optical density (A₂₈₀) falls to background, then the protein is eluted with 0.23 M glycine-HCl (pH 2.6). The purified human nNR1, nNR2 and/or nNR2-1 protein is then dialyzed against phosphate buffered saline.

Levels of human nNR1, nNR2 and/or nNR2-1 in host cells is quantified by a variety of techniques including, but not limited to, immunoaffinity and/or ligand affinity techniques. nNR1, nNR2 and/or nNR2-1-specific affinity beads or nNR1, nNR2 and/or nNR2-1-specific antibodies are used to isolate ³⁵S-methionine labeled or unlabelled nNR1, nNR2 and/or nNR2-1. Labeled nNR1, nNR2 and/or nNR2-1 protein is analyzed by SDS-PAGE. Unlabelled nNR1, nNR2 and/or nNR2-1 protein is detected by Western blotting, ELISA or RIA assays employing either nNR1, nNR2 and/or nNR2-1 protein specific antibodies and/or antiphosphotyrosine antibodies.

Following expression of nNR1, nNR2 and/or nNR2-1 in a host cell, nNR1, nNR2 and/or nNR2-1 protein may be recovered to provide nNR1, nNR2 and/or nNR2-1 protein in active form. Several nNR1, nNR2 and/or nNR2-1 protein purification procedures are available and suitable for use. Recombinant nNR1, nNR2 and/or nNR2-1 protein may be purified from cell lysates and extracts, or from conditioned culture medium, by various combinations of, or individual application of salt fractionation, ion exchange chromatography, size exclusion chromatography, hydroxylapatite adsorption chromatography and hydrophobic interaction chromatography.

The present invention is also directed to methods for screening for compounds which modulate the expression of DNA or RNA encoding a human nNR1, nNR2 and/or nNR2-1 protein. Compounds which modulate these activities may be DNA, RNA, peptides, proteins, or non-proteinaceous organic molecules. Compounds may modulate by increasing or attenuating the expression of DNA or RNA encoding human nNR1, nNR2 and/or nNR2-1, or the function of human nNR1, nNR2 and/or nNR2-1. Compounds that modulate the expression of DNA or RNA encoding human nNR1, nNR2 and/or nNR2-1 or the biological function thereof may be detected by a variety of assays. The assay may be a simple “yes/no” assay to determine whether there is a change in expression or function. The assay may be made quantitative by comparing the expression or function of a test sample with the levels of expression or function in a standard sample. Kits containing human nNR1, nNR2 and/or nNR2-1, antibodies to human nNR1, nNR2 and/or nNR2-1, or modified human nNR1, nNR2 and/or nNR2-1 may be prepared by known methods for such uses.

The DNA molecules, RNA molecules, recombinant protein and antibodies of the present invention may be used to screen and measure levels of human nNR1, nNR2 and/or nNR2-1. The recombinant proteins, DNA molecules, RNA molecules and antibodies lend themselves to the formulation of kits suitable for the detection and typing of human nNR1, nNR2 and/or nNR2-1. Such a kit would comprise a compartmentalized carrier suitable to hold in close confinement at least one container. The carrier would further comprise reagents such as recombinant nNR1, nNR2 and/or nNR2-1 or anti-nNR1, nNR2 and/or nNR2-1 antibodies suitable for detecting human nNR1, nNR2 and/or nNR2-1. The carrier may also contain a means for detection such as labeled antigen or enzyme substrates or the like.

Pharmaceutically useful compositions comprising modulators of human nNR1, nNR2 and/or nNR2-1 may be formulated according to known methods such as by the admixture of a pharmaceutically acceptable carrier. Examples of such carriers and methods of formulation may be found in Remington's Pharmaceutical Sciences. To form a pharmaceutically acceptable composition suitable for effective administration, such compositions will contain an effective amount of the protein, DNA, RNA, modified human nNR1, nNR2 and/or nNR2-1, or either nNR1, nNR2 and/or nNR2-1 agonists or antagonists.

Therapeutic or diagnostic compositions of the invention are administered to an individual in amounts sufficient to treat or diagnose disorders. The effective amount may vary according to a variety of factors such as the individual's condition, weight, sex and age. Other factors include the mode of administration.

The pharmaceutical compositions may be provided to the individual by a variety of routes such as subcutaneous, topical, oral and intramuscular.

The term “chemical derivative” describes a molecule that contains additional chemical moieties which are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

Compounds identified according to the methods disclosed herein may be used alone at appropriate dosages. Alternatively, co-administration or sequential administration of other agents may be desirable.

The present invention also has the objective of providing suitable topical, oral, systemic and parenteral pharmaceutical formulations for use in the novel methods of treatment of the present invention. The compositions containing compounds identified according to this invention as the active ingredient can be administered in a wide variety of therapeutic dosage forms in conventional vehicles for administration. For example, the compounds can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

For combination treatment with more than one active agent, where the active agents are in separate dosage formulations, the active agents can be administered concurrently, or they each can be administered at separately staggered times.

The dosage regimen utilizing the compounds of the present invention is selected in accordance with a variety of factors including type, species, age, weight, sex and medical condition of the patient; the severity of the condition to be treated; the route of administration; the renal, hepatic and cardiovascular function of the patient; and the particular compound thereof employed. A physician or veterinarian of ordinary skill can readily determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug.

The following examples are provided to illustrate the present invention without, however, limiting the same hereto.

### EXAMPLE 1

### Isolation and Characterization of DNA Fragments Encoding nNR1, nNR2 and/or nNR2-1

The DNA sequences from several representative subfamilies (Giguère, et al., 1988, Nature 331: 91-94) were used to query the EST database by using the Blastn program. Two ESTs (Genbank accession number h91890 (nNR1) and w26275 (nNR2)) were identified with homology to human ERR2 at DNA sequence level.

EST h91890 is disclosed herein as SEQ ID NO:7 and is as set forth:

| | |
|---|---|
| CTTTTTAGGA GGTGGAGAAA TTTGTAAGCT CAGGTATGGG CTGCTCTCTG | (SEQ ID NO:7) |
| AGTCCAGCCG TCGCTTGTAT TTCTGACGGC CTCCACGCAC TCGATCAAGG | |
| CGCACACCTT CCTTCAGCAT CCCCACTTTG AGGCATTTCA TGAAGCGGCA | |
| GGCCTGGCAG GACTTGCGCC TCCGTTTGGT GATCTCGCAC TCGTTGGTGG | |
| CCGGGCAGCT GTACTCAATG TTCCCTTGGA TAGTCCTCTT GAAGAAGGCC | |
| TTGCAAGCCT CGCAGGAGGC CCACGCGTNA GTGGTAGCCA GAGNAAATGT | |
| CCCCGCACAC GAGGCACAGG CGCTTGGGGA TGGCGTTGAG CATGTTACTT | |
| CGCACTTGGA TGGGCCGAGT CCTCCATGGA TGGCCGCTGG CAACAGTTCC | |
| TCG. | |

EST w26275 is disclosed herein as SEQ ID NO:8 and is as set forth:

| | |
|---|---|
| CNNNNNNNNN NNNTTTTNNT GCCTAAAGTG GTACCCNGAA GNGATGTCAC | (SEQ ID NO:8) |
| CACACACTAA ACACAGTCTC TTGGGCATCG AGTTGAGCAT GTATTCACAC | |
| TTGGTCTGGG GATCTTCAAC AATGGTGCTG GAGCAGTCAT CATACAGTTT | |
| CCTGACAGGC CCACTACCTC CCAGGATAGG AGCAGAAGGG TAGAGAGGTG | |
| GCGAGTCAAG TCCGTTCTGA TGGCCATTCA TGGTTGAACT GTAGCTCCCA | |
| CTGGCGTCTG AAGAGCCACC AGGGCTGTGG TGGTTGACGC TGTCCGTCAG | |
| GGAGGCTGGG CTGGAAGGTT CCGTCTTGAT GAAGGACGAA CAGCTGGAAT | |
| CAATGTGTCG ATCTTTGTTT GGACATTCTG CAGAGAAGCT CTTCCTCCGT | |
| NGTGCAGGGA AAAAGATTCA GGAAGGCAAA GTTCTTCCCG AATCCATGTG | |
| CGACCGGAAA CCATTATTTG NGCACCCCAG CTATTAATCA AAGTTCCTTG | |
| ACAGAGACAG GGCAATTACA NAATGTCTCC TNTNGGGGAT CAACTGTTCN | |
| GTATTNNNNN NNNNNNNNNN NNNNNNNNNN NNNNNNNNNN NNNNNNNNNN | |
| NNNNNNNNNN NNNNNNNNNN TT. | |

Primer pairs 5′-TGAGTCCAGCCGTCGCTTGTAT-3′ (ERR4F1; SEQ ID NO:9), 5′-TGCAAGCCTCGCAGGAGGCC-3′ (ERR4iF1; SEQ ID NO:10), and 5′-GGCCTTCTTCAAGAGGACTATC-3′(ERR4R1; SEQ ID NO:11) were designed from h91890; 5′-AAAGATCGACACATTGATTCC-3′ (ERR5F; SEQ ID NO:12), 5′-GACTTGACTCGCCACCTCTC-3′ (ERR5iF; SEQ ID NO:13) and 5′-GTTCTGATGGCCATTCATGGT-3′ (ERR5R; SEQ ID NO:14) were designed from W26275. Primer pairs ERR4F/ERR4R and ERR5F/RR5R were used to scan cDNA made from testis, fetal brain, prostate and placenta first before scanning cDNA libraries made from those cDNA and distributed in 96-well plates. Primers for nNR1 produced a PCR product from testis cDNA, while primers for nNR2 generated a PCR product a cDNA library generated from fetal brain, prostate and placenta mRNA. Therefore, a cDNA library made from testis with >2.5 kb insert was used for nNR1 positive pool identification, and A4 and G8 gave the PCR product of expected size. Inverse PCR using ERR4iF1 and ERR4R1 were performed on positive pools and DNA fragments of about 6.0 kb were amplified. The DNA fragment was purified using Qiagen gel extraction kit. Phosphorylation, self-ligation and transformation of the purified DNA was carried out. DNA mini-preps from four individual clones were used in automated sequencing with gene specific and vector primers. Since a PCR-induced mutation is possible in long PCR reactions, nNR1 was re-subcloned in to the PCR2.1 vector (Invitrogen) using a PCR fragment amplified by a 5′-primer 5′-GAATATGATGACCCTAATGCA-3′ (SEQ ID NO:15) and a 3′-primer 5′-CTTCCACCTCATGGACACCAA-3′ (SEQ ID NO:16) on the positive A4 pool. One out of the four TA-clones showed no mutation through sequencing confirmation. DNA sequence analysis was performed using the ABI PRISM™ dye terminator cycle sequencing ready reaction kit with AmpliTaq DNA polymerase, FS (Perkin Elmer, Norwalk, Cont.). DNA sequence analysis was performed with M13 forward/reverse primers and gene specific sequencing primers manufactured by GIBCO BRL (Gaithersburg, Md.). Sequence assembly and analysis were performed with SEQUENCHER™ 3.0 (Gene Codes Corporation, Ann Arbor, Minn.). Ambiguities and/or discrepancies between automated base calling in sequencing reads were visually examined and edited to the correct base call. Several regions were resequenced after initial automated or visual calling. Four oligonucleotides close to the regions with potential sequence ambiguities were utilized ([R1F1] 5′-CAT TCC ACG GAG GCA TCC TC-3′ (SEQ ID NO:23); [R1F2] 5′-CCA AGG CCG TGC AGC ACT TC-3′ (SEQ ID NO:24); [R1R1] 5′-GAC AGC CTC TAG ATC CTC GAT-3′ (SEQ ID NO:25); and, [R1R2] 5′-ATC ATG GCT TGA CAT TCT TTC-3′ (SEQ ID NO:26) and automated sequencing was performed. The final nucleotide sequence encoding NR1 is shown as set forth in FIGS. 1A-C and as set forth as SEQ ID NO:1.

For nNR2, a cDNA library made from fetal brain with >2.5 kb insert was used. Positive pools C1, F7 and G6 were identified and used in inverse PCR with primer pairs ERR5iF/ERR5R. A PCR fragment of ˜6.0 kb was amplified from C1. The same methodology as described herein for nNR1 was applied to isolation, characterization and sequencing of a nNR2 cDNA. The cDNA fragment cloned into pCR2.1 vector was amplified by 5′-primer 5′-GTTAATTGCACTGTGCTCTG-3′ (SEQ ID NO:17) and 3′-primer 5′-AGTGTGGTGGAATTCTCTTA-3′ (SEQ ID NO:18).

Primer pairs XR2F3 (5′-AGCTCTTGCTAATTCAGAC-3′ [SEQ ID NO:27]) and XR2R4 (5′-TCAACATGAAGGATGGGAAGG-3′ [SEQ ID NO:28]) were used in DNA sequence analysis (performed using the ABI PRISM™ dye terminator cycle sequencing ready reaction kit with AmpliTaq DNA polymerase, FS (Perkin Elmer, Norwalk, Cont.)) of the carboxy region of nNR2. DNA sequence analysis was performed with M13 forward/reverse primers and gene specific sequencing primers customarily manufactured by GIBCO BRL (Gaithersburg, Md.). Sequence assembly and analysis were performed with SEQUENCHER™ 3.0 (Gene Codes Corporation, Ann Arbor, Minn.). Ambiguities and/or discrepancies between automated base calling in sequencing reads were visually examined and edited to the correct base call. Resequencing of the ligand binding domain showed a new open reading frame that was confirmed with the XR2F3/XR2R4 primers. The nNR2 peptide coded by the complete open reading frame has 40 extra amino acids at C-terminus compared to nNR2-1 and is similar in length to its closest related member hERR2.

In order to identify the genome map position of the genes, primers in the 3′ non-coding region were designed. Forwarding primer 5′-TCTAGTGTTGCTGCGAGTGAC-3′ (SEQ ID NO:19) and reversing primer 5′-CTTCCACCTCATGGACACCAA-3′ (SEQ ID NO:20) were used for nNR1, while forwarding primer 5′-GTCTGACTAAAAGCTCCCTG-3′ (SEQ ID NO:21) and reversing primer 5′-GAAGATGATGGAGAAAGTAGA-3′ (SEQ ID NO:22) were used for nNR2. PCR scanning was performed on the 83 clones of the Stanford radiation hybrid panel (Cox et al., 1990, Science, 250:245:250). The PCR results were scored and submitted to the Stanford Genome Center for linkage analysis. The results indicate that nNR1 is located on locus 14q24.3˜14q31 and nNR2 is located on chromosome 1.

What is claimed:

1. A purified DNA molecule encoding a human nNR1 protein wherein said protein comprises the amino acid sequence as follows:
MSSDDRHLGS SCGSFIKTEP SSPSSGIDAL SHHSPSGSSD
ASGGFGLALG THANGLDSPP MFAGAGLGGT PCRKSYEDCA
SGIMEDSAIK CEYMLNAIPK RLCLVCGDIA SGYHYGVASC
EACKAFFKRT IQGNIEYSCP ATNECEITKR RRKSCQACRF
MKCLKVGMLK EGVRLDRVRG GRQKYKRRLD SESSPYLSLQ
ISPPAKKPLT KIVSYLLVAE PDKLYAMPPP GMPEGDIKAL
TTLCDLADRE LVVIIGWAKH IPGFSSLSLG DQMSLLQSAW
MEILILGIVY RSLPYDDKLV YAEDYIMDEE HSRLAGLLEL
YRAILQLVRR YKKLKVEKEE FVTLKALALA NSDSMYIEDL
EAVQKLQDLL HEALQDYELS QRHEEPWRTG KLLLTLPLLR
QTAAKAVQHF YSVKLQGKVP MHKLFLEMLE AKAWARADSL
QEWRPLEQVP SPLHRATKRQ HVHFLTPLPP PPSVAWVGTA
QAGYHLEVFL PQRAGWPRAA,

2. An expression vector for expressing a human nNR1 protein in a recombinant host cell wherein said expression vector comprises a DNA molecule of claim 1.

3. A host cell which expresses a recombinant human nNR1 protein wherein said host cell contains the expression vector of claim 2.

4. A process for expressing a human nNR1 protein in a recombinant host cell, comprising:
(a) transfecting the expression vector of claim 2 into a suitable host cell; and,
(b) culturing the host cells of step (a) under conditions which allow expression of said the human nNR1 protein from said expression vector.

5. A purified DNA molecule encoding a human nNR1 protein wherein said protein consists of the amino acid sequence as follows:
MSSDDRHLGS SCGSFIKTEP SSPSSGIDAL SHHSPSGSSD
ASGGFGLALG THANGLDSPP MFAGAGLGGT PCRKSYEDCA
SGIMEDSAIK CEYMLNAIPK RLCLVCGDIA SGYHYGVASC
EACKAFFKRT IQGNIEYSCP ATNECEITKR RRKSCQACRF
MKCLKVGMLK EGVRLDRVRG GRQKYKRRLD SESSPYLSLQ
ISPPAKKPLT KIVSYLLVAE PDKLYAMPPP GMPEGDIKAL
TTLCDLADRE LVVIIGWAKH IPGFSSLSLG DQMSLLQSAW
MEILILGIVY RSLPYDDKLV YAEDYIMDEE HSRLAGLLEL
YRAILQLVRR YKKLKVEKEE FVTLKALALA NSDSMYIEDL
EAVQKLQDLL HEALQDYELS QRHEEPWRTG KLLLTLPLLR
QTAAKAVQHF YSVKLQGKVP MHKLFLEMLE AKAWARADSL
QEWRPLEQVP SPLHRATKRQ HVHFLTPLPP PPSVAWVGTA
QAGYHLEVFL PQRAGWPRAA,

6. An expression vector for expressing a human nNR1 protein in a recombinant host cell wherein said expression vector comprises a DNA molecule of claim 5.

7. A host cell which expresses a recombinant human nNR1 protein wherein said host cell contains the expression vector of claim 6.

8. A process for expressing a human nNR1 protein in a recombinant host cell, comprising:
(a) transfecting the expression vector of claim 6 into a suitable host cell; and,
(b) culturing the host cells of step (a) under conditions which allow expression of said the human nNR1 protein from said expression vector.

9. A purified DNA molecule encoding a human nNR1 protein wherein said DNA molecule comprises the nucleotide sequence as set forth in SEQ ID NO:1, as follows:

| | |
|---|---|
| GAATATGATG ACCCTAATGC AACAATATCT | (SEQ ID NO:1) |
| AACATACTAT CCGAGCTTCG GTCATTTGGA | |
| AGAACTGCAG ATTTTCCTCC TTCAAAATTA | |
| AAGTCAGCTT ATGGAGAACA TGTATGCTAT | |
| GTTCTTGATT GCTTCGCTGA AGAAGCATTG | |
| AAATATATTG GTTTCACCTG GAAAAGGCCA | |
| ATATACCCAG TAGAAGAATT AGAAGAAGAA | |
| AGCGTTGCAG AAGATGATGC AGAATTAACA | |
| TTAAATAAAG TGGATGAAGA ATTTGTGGAA | |
| GAAGAGACAG ATAATGAAGA AAACTTTATT | |
| GATCTCAACG TTTTAAAGGC CCAGACATAT | |
| CACTTGGATA TGAACGAGAC TGCCAAACAA | |
| GAAGATATTT TGGAATCCAC AACAGATGCT | |
| GCAGAATGGA GCCTAGAAGT GGAACGTGTA | |
| CTACCGCAAC TGAAAGTCAC GATTAGGACT | |
| GACAATAAGG ATTGGAGAAT CCATGTTGAC | |
| CAAATGCACC AGCACAGAAG TGGAATTGAA | |
| TCTGCTCTAA AGGAGACCAA GGGATTTTTG | |
| GACAAACTCC ATAATGAAAT TACTAGGACT | |
| TTGGAAAAGA TCAGCAGCCG AGAAAAGTAC | |
| ATCAACAATC AGCCGGGAGC CCATGGAGCA | |
| CTGTCCTCAG AGATGCGCAG GTTAGGCTCA | |
| CTGTCTAGGC CAGGCCCACC TTAGTCACTG | |
| TGGACTGGCA ATGGAAGCTC TTCCTGGACA | |
| CACCTGCCCT AGCCCTCACC CTGGGGTGGA | |
| AGAGAAATGA GCTTGGCTTG CAACTCAGAC | |
| CATTCCACGG AGGCATCCTC CCCTTCCCTG | |
| GGCTGGTGAA TAAAAGTTTC CTGAGGTCAA | |
| GGACTTCCTT TTCCCTGCCA AAATGGTGTC | |
| CAGAACTTTG AGGCCAGAGG TGATCCAGTG | |
| ATTTGGGAGC TGCAGGTCAC ACAGGCTGCT | |
| CAGAGGGCTG CTGAACAGGA TGTCCTCGGA | |
| CGACAGGCAC CTGGGCTCCA GCTGCGGCTC | |
| CTTCATCAAG ACTGAGCCGT CCAGCCCGTC | |
| CTCGGGCATA GATGCCCTCA GCCACCACAG | |
| CCCCAGTGGC TCGTCCGACG CCAGCGGCGG | |
| CTTTGGCCTG GCCCTGGGCA CCCACGCCAA | |
| CGGTCTGGAC TCGCCACCCA TGTTTGCAGG | |
| CGCCGGGCTG GGAGGCACCC CATGCCGCAA | |
| GAGCTAGGAG GACTGTGCCA GCGGCATCAT | |
| GGAGGACTCG GCCATCAAGT GCGAGTACAT | |
| GCTCAACGCC ATCCCCAAGC GCCTGTGCCT | |
| CGTGTGCGGG GACATTGCCT CTGGCTACCA | |
| CTACGGCGTG GCCTCCTGCG AGGCTTGCAA | |
| GGCCTTCTTC AAGAGGACTA TCCAAGGGAA | |
| CATTGAGTAC AGCTGCCCGG CCACCAACGA | |
| GTGCGAGATC ACCAAACGGA GGCGCAAGTC | |
| CTGCCAGGCC TGCCGCTTCA TGAAATGCCT | |
| CAAAGTGGGG ATGCTGAAGG AAGGTGTGCG | |
| CCTTGATCGA GTGCGTGGAG GCCGTCAGAA | |
| ATACAAGCGA CGGCTGGACT CAGAGAGGAG | |
| CCCATACCTG AGCTTACAAA TTTCTCCACC | |
| TGCTAAAAAG GGATTGAGGA AGATTGTGTG | |
| ATACGTAGTG GTGGCTGAGC CGGACAAGCT | |
| CTATGCCATG CCTCCCCCTG GTATGCCTGA | |
| GGGGGACATC AAGGCCCTGA CCACTCTCTG | |
| TGACCTGGCA GACCGAGAGC TTGTGGTCAT | |
| CATTGGCTGG GCCAAGCACA TCCCAGGCTT | |
| CTCAAGCCTC TCCCTGGGGG ACCAGATGAG | |
| CCTGCTGCAG AGTGCCTGGA TGGAAATCCT | |
| CATCCTGGGC ATCGTGTACC GCTCGCTGCC | |
| CTACGACGAC AAGCTGGTGT ACGCTGAGGA | |
| CTACATCATG GATGAGGAGC ACTCCCGCCT | |
| CGCGGGGCTG CTGGAGCTCT ACCGGGCCAT | |
| CCTGCAGCTG GTACGCAGGT ACAAGAAGCT | |
| CAAGGTGGAG AAGGAGGAGT TTGTGACGCT | |
| CAAGGCCCTG GCCCTCGCCA ACTCCGATTC | |
| CATGTACATC GAGGATCTAG AGGCTGTCCA | |
| GAAGCTGCAG GACCTGCTGC ACGAGGCACT | |
| GCAGGACTAC GAGCTGAGCC AGCGCCATGA | |
| GGAGCCCTGG AGGACGGGCA AGCTGCTGCT | |
| GACACTGCCG CTGCTGCGGC AGACGGCCGC | |
| CAAGGCCGTG CAGCACTTCT ATAGCGTCAA | |
| ACTGCAGGGC AAAGTGCCCA TGCACAAACT | |
| CTTCCTGGAG ATGCTGGAGG CCAAGGCCTG | |
| GGCCAGGGCT GACTCCCTTC AGGAGTGGAG | |
| GCCACTGGAG CAAGTGCCCT CTCCCCTCCA | |
| CCGAGCCACC AAGAGGCAGC ATGTGCATTT | |
| CCTAACTCCC TTGCCCCCTC CCCCATCTGT | |
| GGCCTGGGTG GGCACTGCTC AGGCTGGATA | |
| CCACCTGGAG GTTTTCCTTC CGCAGAGGGC | |
| AGGTTGGCCA AGAGCAGCTT AGAGGATCTC | |
| CCAAGGATGA AAGAATGTCA AGCCATGATG | |
| GAAAATGCCC CTTCCAATCA GCTGCCTTCA | |
| CAAGCAGGGA TCAGAGCAAC TCCCCGGGGA | |
| TCCCCAATCC ACGCCCTTCT AGTCCAACCC | |
| CCCTCAATGA GAGAGGCAGG CAGATCTCAC | |
| CCAGCACTAG GACACCAGGA GGCCAGGGAA | |
| AGCATCTCTG GCTCACCATG TAACATCTGG | |
| CTTGGAGCAA GTGGGTGTTC TGCACACCAG | |
| GCAGCTGCAC CTCACTGGAT CTAGTGTTGC | |
| TGCGAGTGAC CTCACTTCAG AGCCCCTCTA | |
| GCAGAGTGGG GCGGAAGTCC TGATGGTTGG | |
| TGTCCATGAG GTGGAAG. | |

10. A DNA molecule of claim 9 which comprises from about nucleotide 950 to about nucleotide 2452 of SEQ ID NO:1.

11. An expression vector for expressing a human nNR1 protein wherein said expression vector comprises a DNA molecule of claim 9.

12. An expression vector for expressing a human nNR1 protein wherein said expression vector comprises a DNA molecule of claim 11.

13. A host cell which expresses a recombinant human nNR1 protein wherein said host cell contains the expression vector of claim 11.

14. A host cell which expresses a recombinant human nNR1 protein wherein said host cell contains the expression vector of claim 12.

15. A process for expressing a human nNR1 protein in a recombinant host cell, comprising:
(a) transfecting the expression vector of claim 11 into a suitable host cell; and,
(b) culturing the host cells of step (a) under conditions which allow expression of said the human nNR1 protein from said expression vector.

16. A puified DNA molecule encoding a human nNR1 protein wherein said DNA molecule consists of the nucleotide sequence as set forth in SEQ ID NO:1, as follows:

| | |
|---|---|
| GAATATCATG ACCCTAATGC AACAATATCT | (SEQ ID NO:1) |
| AACATACTAT CCGAGCTTCG GTCATTTGGA | |
| AGAACTGCAG ATTTTCCTCC TTCAAAATTA | |
| AAGTCAGGTT ATGGAGAACA TGTATGCTAT | |
| GTTCTTGATT GCTTCGCTGA AGAAGCATTG | |
| AAATATATTG GTTTCACCTG GAAAAGGCCA | |
| ATATACCCAG TAGAAGAATT AGAAGAAGAA | |
| AGCGTTGCAG AAGATGATGC AGAATTAACA | |
| TTAAATAAAG TGGATGAAGA ATTTGTGGAA | |
| GAAGAGACAG ATAATGAAGA AAACTTTATT | |
| GATCTCAACG TTTTAAAGGC CCAGACATAT | |
| CACTTGGATA TGAACGAGAC TGCCAAACAA | |
| GAAGATATTT TGGAATCCAC AACAGATGCT | |
| GCAGAATGGA GCCTAGAAGT GGAACGTGTA | |
| CTACCGCAAC TGAAAGTCAC GATTAGGACT | |
| GACAATAAGG ATTGGAGAAT CCATGTTGAC | |
| CAAATGCACC AGCACAGAAG TGGAATTGAA | |
| TCTGCTCTAA AGGAGACCAA GGGATTTTTG | |
| GACAAACTCC ATAATGAAAT TACTAGGACT | |
| TTGGAAAAGA TCAGCAGCCG AGAAAAGTAC | |
| ATCAACAATC AGCCGGGAGC CCATGGAGCA | |
| CTGTCCTCAG AGATGCGCAG GTTAGGCTCA | |
| CTGTCTAGGC CAGGCCCACC TTAGTCACTG | |
| TGGACTGGCA ATGGAAGCTC TTCCTGGACA | |
| CACCTGCCCT AGCCCTCACC CTGGGGTGGA | |
| AGAGAAATGA GCTTGGCTTG CAACTCAGAC | |
| CATTCCACGG AGGCATCCTC CCCTTCCCTG | |
| GGCTGGTGAA TAAAAGTTTC CTGAGGTCAA | |
| GGACTTCCTT TTCCCTGCCA AAATGGTGTC | |
| CAGAACTTTG AGGCCAGAGG TGATCCAGTG | |
| ATTTGGGAGC TGCAGGTCAC ACAGGCTGCT | |
| CAGAGGOCTG CTGAACAGGA TGTCCTCGGA | |
| CGACAGGCAC CTGGGCTCCA GCTGCGGCTC | |
| CTTCATCAAG ACTGAGCCGT CCAGCCCGTC | |
| CTCGGGCATA GATGCCCTCA GCCACCACAG | |
| CCCCAGTGGC TCGTCCGACG CCAGCGGCGG | |
| CTTTGGCCTG GCCCTGGGCA CCCACGCCAA | |
| CGGTCTGGAC TCGCCACCCA TGTTTGCAGG | |
| CGCCGGGCTG GGAGGCACCC CATGCCGCAA | |
| GAGCTACGAG GACTGTGCCA GCGGCATCAT | |
| GGAGGACTCG GCCATCAAGT GCGAGTACAT | |
| GCTCAACGCC ATCCCCAAGC GCCTGTGCCT | |
| CGTGTGCGGG GACATTGCCT CTGGCTACCA | |
| CTACGGCGTG GCCTCCTGCG AGGCTTGCAA | |
| GGCCTTCTTC AAGAGGACTA TCCAAGGGAA | |
| CATTGAGTAC AGCTGCCCGG CCACCAACGA | |
| GTGCGAGATC ACCAAACGGA GGCGCAAGTC | |
| CTGCCAGGCC TGCCGCTTCA TGAAATGCCT | |
| CAAAGTGGGG ATGCTGAAGG AAGGTGTGCG | |
| CCTTGATCGA GTGCGTGGAG GCCGTCAGAA | |
| ATACAAGCGA CGGCTGGACT CAGAGAGCAG | |
| CCCATACCTG AGCTTACAAA TTTCTCCACC | |
| TGCTAAAAAG CCATTGACCA AGATTGTCTC | |
| ATACCTACTG GTGGCTGAGC CGGACAAGCT | |
| CTATGCCATG CCTCCCCCTG GTATGCCTGA | |
| GGGGGACATC AAGGCCCTGA CCACTCTCTG | |
| TGACCTGGCA GACCGAGAGC TTCTGGTCAT | |
| CATTGGCTGG GCCAAGCACA TCCCAGGCTT | |
| CTCAAGCCTC TCCCTGGGGG ACCAGATGAG | |
| CCTGCTGCAG AGTGCCTGGA TGGAAATCCT | |
| CATCCTGGGC ATCGTGTACC GCTCGCTGCC | |
| CTACGACGAC AAGCTGGTGT ACGCTGAGGA | |
| CTACATCATG GATOAGGAGC ACTCCCGCCT | |
| CGCGGGGCTG CTGGAGCTCT ACCGGGCCAT | |
| CCTGCAGCTG GTACGCAGGT ACAAGAAGCT | |
| CAAGGTGGAG AAGGAGGAGT TTGTGACGCT | |
| CAAGGCCCTG GCCCTCGCCA ACTCCGATTC | |
| CATGTACATC GAGGATCTAG AGGCTGTCCA | |
| GAAGCTGCAG GACCTGCTGC ACGAGGCACT | |
| GCAGGACTAC GAGCTGAGCC AGCGCCATGA | |
| GGAGCCCTGG AGGACGGGCA AGCTGCTGCT | |
| GACACTGCCG CTGCTGCGGC AGACGGCCGC | |
| CAAGGCCGTG CAGCACTTCT ATAGCGTCAA | |
| ACTGCAGGGC AAAGTGCCCA TGCACAAACT | |
| CTTCCTGGAG ATGCTGGAGG CCAAGGCCTG | |
| GGCCAGGGCT GACTCCCTTC AGGAGTGGAG | |
| GCCACTGGAG CAAGTGCCCT CTCCCCTCCA | |
| CCGAGCCACC AAGAGGCAGC ATGTGCATTT | |
| CCTAACTCCC TTGCCCCCTC CCCCATCTGT | |
| GGCCTGGGTG GGCACTGCTC AGGCTGGATA | |
| CCACCTGGAG GTTTTCCTTC CGCAGAGGGC | |
| AGGTTGGCCA AGAGCAGCTT AGAGGATCTC | |
| CCAAGGATGA AAGAATGTCA AGCCATGATG | |
| GAAAATGCCC CTTCCAATCA GCTGCCTTCA | |
| CAAGCAGGGA TCAGAGCAAC TCCCCGGGGA | |
| TCCCCAATCC ACGCCCTTCT AGTCCAACCC | |
| CCCTCAATGA GAGAGGCAGG CAGATCTCAC | |
| CCAGCACTAG GACACCAGGA GGCCAGGGAA | |
| AGCATCTCTG GCTCACCATG TAACATCTGG | |
| CTTGGAGCAA GTGGGTGTTC TGCACACCAG | |
| GCAGCTGCAC CTCACTGGAT CTAGTGTTGC | |
| TGCGAGTGAC CTCACTTCAG AGCCCCTCTA | |
| GCAGAGTGGG GCGGAAGTCC TGATGGTTGG | |
| TGTCCATGAG GTGGAAG. | |

17. A DNA molecule of claim 16 which consists of nucleotide 950 to about nucleotide 2452 of SEQ ID NO:1.

18. An expression vector for expressing a human nNR1 protein wherein said expression vector comprises a DNA molecule of claim 16.

19. An expression vector for expressing a human nNR1 protein wherein said expression vector comprises a DNA molecule of claim 17.

20. A host cell which expresses a recombinant human nNR1 protein wherein said host cell contains the expression vector of claim 18.

21. A host cell which expresses a recombinant human nNR1 protein wherein said host cell contains the expression vector of claim 19.

22. A process for expressing a human nNR1 protein in a recombinant host cell, comprising:
(a) transfecting the expression vector of claim 18 into a suitable host cell; and,
(b) culturing the host cells of step (a) under conditions which allow expression of said the human nNR1 protein from said expression vector.

23. A purified DNA molecule encoding a human nNR2 protein wherein said protein comprises the amino acid sequence as follows:

| | |
|---|---|
| MDSVELCLPE SFSLHYEEEL LCRMSNKDRH | SEQ ID NO:4 |
| IDSSCSSFIK TEPSSPASLT DSVNHHSPGG | |
| SSDASGSYSS TMNGHQNGLD SPPLYPSAPI | |
| LGGSGPVRKL YDDCSSTIVE DPQTKCEYML | |
| NSMPKRLCLV CGDIASGYHY GVASCEACKA | |
| FFKRTIQGNI EYSCPATNEC EITKRRRKSC | |
| QACRFMKCLK VGMLKEGVRL DRVRGGRQKY | |
| KRRIDAENSP YLNPQLVQPA KKPYNKIVSH | |
| LLVAEPEKIY AMPDPTVPDS DIKALTTLCD | |
| LADRELVVII GWAKHIPGFS TLSLADQMSL | |
| LQSAWMEILI LGVVYRSLSF EDELVYADDY | |
| IMDEDQSKLA GLLDLNNAIL QLVKKYKSMK | |
| LEKEEFVTLK AIALANSDSM HIEDVEAVQK | |
| LQDVLHEALQ DYEAGQHMED PRRAGKMLMT | |
| LPLLRQTSTK AVQHFYNIKL EGKVPNHKLF | |
| LEMLEAIKV, as set forth in three-letter | |
| abbreviation in. | |

24. An expression vector for expressing a human nNR2 protein in a recombinant host cell wherein said expression vector comprises a DNA molecule of claim 23.

25. A host cell which expresses a recombinant human nNR2 protein wherein said host cell contains the expression vector of claim 24.

26. A process for expressing a human nNR2 protein in a recombinant host cell, comprising:
(a) transfecting the expression vector of claim 24 into a suitable host cell; and,
(b) culturing the host cells of step (a) under conditions which allow expression of said the human nNR1 protein from said expression vector.

27. A purified DNA molecule encoding a human nNR2 protein wherein said protein consists of the amino acid sequence as follows:

| | |
|---|---|
| MDSVELCLPE SFSLHYEEEL LCRMSNKDRH | SEQ ID NO.4 |
| IDSSCSSFIK TEPSSPASLT DSVNHHSPGG | |
| SSDASGSYSS TMNGHQNGLD SPPLYPSAPI | |
| LGGSGPVRKL YDDCSSTIVE DPQTKCEYML | |
| NSMPKRLCLV CGDIASGYHY GVASCEACKA | |
| FFKRTIQGNI EYSCPATNEC EITKRRRKSC | |
| QACRFMKCLK VGMLKEGVRL DRVRGGRQKY | |
| KRRIDAENSP YLNPQLVQPA KKPYNKIVSH | |
| LLVAEPEKIY AMPDPTVPDS DIKALTTLCD | |
| LADRELVVII GWAKHIPGFS TLSLADQMSL | |
| LQSAWMEILI LGVVYRSLSF EDELVYADDY | |
| IMDEDQSKLA GLLDLNNAIL QLVKKYKSMK | |
| LEKEEFVTLK ATALANSDSM HIEDVEAVQK | |
| LQDVLHEALQ DYEAGQHMED PRRAGKMLMT | |
| LPLLRQTSTK AVQHFYNIKL EGKVPNHKLF | |
| LEMLEAKV, as set forth in three letter | |
| code as. | |

28. An expression vector for expressing a human nNR2 protein in a recombinant host cell wherein said expression vector comprises a DNA molecule of claim 27.

29. A host cell which expresses a recombinant human nNR1 protein wherein said host cell contains the expression vector of claim 28.

30. A process for expressing a human nNR2 protein in a recombinant host cell, comprising:
(a) transfecting the expression vector of claim 28 into a suitable host cell; and,
(b) culturing the host cells of step (a) under conditions which allow expression of said the human nNR1 protein from said expression vector.

31. A purified DNA molecule encoding a human nNR2 protein wherein said DNA molecule comprises the nucleotide sequence as set forth in SEQ ID NO:3, as follows:

| | |
|---|---|
| GCGGGCCGCC AGTGTGGTGG AATTCGGCTT | (SEQ ID NO:3) |
| GTCACTAGGA GAACATTTGT GTTAATTGCA | |
| CTGTGCTCTG TCAAGGAAAC TTTGATTTAT | |
| AGCTGGGGTG CACAAATAAT GGTTGCCGGT | |
| CCCACATGGA TTCGGTAGAA CTTTGCCTTC | |
| CTGAATCTTT TTCCCTGCAC TACGAGGAAG | |
| AGCTTCTCTG CAGAATGTCA AACAAAGATC | |
| GACACATTGA TTCCAGCTGT TCGTCCTTCA | |
| TCAAGACGGA ACCTTCCAGC CCAGCCTCCC | |
| TGACGGACAG CGTCAACCAC CACAGCCCTG | |
| GTGGCTCTTC AGACGCCAGT GGGAGCTACA | |
| GTTCAACCAT GAATGGCCAT CAGAACGGAC | |
| TTGACTGGCC ACCTCTCTAC CCTTCTGCTC | |
| CTATCCTGGG AGGTAGTGGG CCTGTCAGGA | |
| AACTGTATGA TGACTGCTCC AGCACCATTG | |
| TTGAAGATCC CCAGACCAAG TGTGAATACA | |
| TGCTCAACTC GATGCCCAAG AGACTGTGTT | |
| TAGTGTGTGG TGACATCGCT TCTGGGTACC | |
| ACTATGGGGT AGCATCATGT GAAGCCTGCA | |
| AGGCATTCTT CAAGAGGACA ATTCAAGGCA | |
| ATATAGAATA CAGCTGCCCT GCCACGAATG | |
| AATGTGAAAT CACAAAGCGC AGACGTAAAT | |
| CCTGCCAGGC TTGCCGCTTC ATGAAGTGTT | |
| TAAAAGTGGG CATGCTGAAA GAAGGGGTGC | |
| GTCTTGACAG AGTACGTGGA GGTCGGCAGA | |
| AGTACAAGCG CAGGATAGAT GCGGAGAACA | |
| GCCCATACCT GAACCCTCAG CTGGTTCAGC | |
| CAGCCAAAAA GCCATATAAC AAGATTGTCT | |
| CACATTTGTT GGTGGCTGAA CCGGAGAAGA | |
| TCTATGCCAT GCCTGACCCT ACTGTCCCCG | |
| ACAGTGACAT CAAAGCCCTC ACTACACTGT | |
| GTGACTTGGC CGACCGAGAG TTGGTGGTTA | |
| TCATTGGATG GGCGAAGCAT ATTCCAGGCT | |
| TCTCCACGCT GTCCCTGGCG GACCAGATGA | |
| GCCTTCTGCA GAGTGCTTGG ATGGAAATTT | |
| TGATCCTTGG TGTCGTATAC CGGTCTCTTT | |
| CATTTGAGGA TGAACTTGTC TATGCAGACG | |
| ATTATATAAT GGACGAAGAC CAGTCCAAAT | |
| TAGCAGGCCT TCTTGATCTA AATAATGCTA | |
| TCCTGCAGCT GGTAAAGAAA TACAAGAGCA | |
| TGAAGCTGGA AAAAGAAGAA TTTGTCACCC | |
| TCAAAGCTAT AGCTCTTGCT AATTCAGACT | |
| CCATGCACAT AGAAGATGTT GAAGCCGTTC | |
| AGAAGCTTCA GGATGTCTTA CATGAAGCGC | |
| TGCAGGATTA TGAAGCTGGC CAGCACATGG | |
| AAGACCCTCG TCGAGCTGGC AAGATCCTGA | |
| TGACACTGCC ACTCCTGAGG CAGACCTCTA | |
| CCAAGGCCGT GCAGCATTTC TACAACATCA | |
| AACTAGAAGG CAAAGTCCCA ATGCACAAAC | |
| TTTTTTTGGA AATGTTGGAG GCCAAGGTCT | |
| GACTAAAAGC TCCCTGGGCC TTCCCATCCT | |
| TCATGTTGAA AAAGGGAAAA TAAACCCAAG | |
| AGTGATGTCG AAGAAACTTA GAGTTTAGTT | |
| AACAACATCA AAAATCAACA GACTGCACTG | |
| ATAATTTAGC AGCAAGACTA TGAAGCAGCT | |
| TTCAGATTCC TCCATAGGTT CCTGATGAGT | |
| TCTTTCTACT TTCTCCATCA TCTTCTTTCC | |
| TCTTTCTTCC CACATTTCTC TTTCTCTTTA | |
| TTTTTTCTCC TTTTCTTCTT TCACCTCCCT | |
| TATTTCTTTG CTTCTTTCAT TCCTAGTTCC | |
| CATTCTCCTT TATTTTCTTC CCGTCTGCCT | |
| GCCTTCTTTC TTTTCTTTAC CTACTCTCAT | |
| TCCTCTCTTT TCTCATCCTT CCCCTTTTTT | |
| CTATATTTGA AATAGCTTTA GTTTAAAAAA | |
| AAAAATCCTC CCTTCCCCCT TTCCTTTCCC | |
| TTTCTTTCCT TTTTCCCTTT CCTTTTCCCT | |
| TTCCTTTCCT TTCCTCTTGA CCTTCTTTCC | |
| ATCTTTCTTT TTCTTCCTTC TGCTGCTGAA | |
| CTTTTAAAAG AGGTCTCTAA CTGAAGAGAG | |
| ATGGAAGCCA GCCCTGCCAA AGGATGGAGA | |
| TCCATAATAT GGATGCCAGT GAACTTATTG | |
| TGAACCATAC CGTCCCCAAT GACTAAGGAA | |
| TCAAAGAGAG AGAACCAACG TTCCTAAAAG | |
| TACAGTGCAA CATATACAAA TTGACTGAGT | |
| GCAGTATTAG ATTTCATGGG AGCAGCCTCT | |
| AATTAGACAA CTTAAGCAAC GTTGCATCGG | |
| CTGCTTCTTA TCATTGCTTT TCCATCTAGA | |
| TCAGTTACAG CCATTTGATT CCTTAATTGT | |
| TTTTTCAAGT CTTCCAGGTA TTTGTTAGTT | |
| TAGCTACTAT GTAACTTTTT CAGGGAATAG | |
| TTTAAGCTTT ATTCATTCAT GCAATACTAA | |
| AGAGAAATAA GAATACTGCA ATTTTGTGCT | |
| GGCTTTGAAC AATTACGAAC AATAATGAAG | |
| GACAAATGAA TCCTGAAGGA AGATTTTTAA | |
| AAATGTTTTG TTTCTTCTTA CAAATGGAGA | |
| TTTTTTTGTA CCAGCTTTAC CACTTTTCAG | |
| CCATTTATTA ATATGGGAAT TTAACTTACT | |
| CAAGCAATAG TTGAAGGGAA GGTGCATATT | |
| ATCACGGATG CAATTTATGT TGTGTGCCAG | |
| TCTGGTCCCA AACATCAATT TCTTAACATG | |
| AGCTCCAGTT TACCTAAATG TTCACTGACA | |
| CAAAGGATGA GATTACACCT ACAGTGACTC | |
| TGAGTAGTCA CATATATAAG CACTGCACAT | |
| GAGATATAGA TCCGTAGAAT TGTCAGGAGT | |
| GCACCTCTCT ACTTGGGAGG TACAATTGCC | |
| ATATGATTTC TAGCTGCCAT GGTGGTTAGG | |
| AATGTGATAC TGCCTGTTTG CAAAGTCACA | |
| GACCTTGCCT CAGAAGGAGC TGTGAGCCAG | |
| TATTCATTTA AGAGAATTCC ACCACACTGG | |
| CGGCCCGCGC TTGAT. | |

32. A DNA molecule of claim 31 which comprises from about nucleotide 126 to about nucleotide 1382 of SEQ ID NO:3.

33. An expression vector for expressing a human nNR2 protein wherein said expression vector comprises a DNA molecule of claim 31.

34. An expression vector for expressing a human nNR2 protein wherein said expression vector comprises a DNA molecule of claim 32.

35. A host cell which expresses a recombinant human nNR2 protein wherein said host cell contains the expression vector of claim 33.

36. A host cell which expresses a recombinant human nNR2 protein wherein said host cell contains the expression vector of claim 34.

37. A process for expressing a human nNR2 protein in a recombinant host cell, comprising:
(a) transfecting the expression vector of claim 33 into a suitable host cell; and,
(b) culturing the host cells of step (a) under conditions which allow expression of said the human nNR1 protein from said expression vector.

38. A purified DNA molecule encoding a human nNR2 protein wherein said DNA molecule consists of the nucleotide sequence as set forth in SEQ ID NO:3, as follows:

| | |
|---|---|
| GCGGGCCGCC AGTGTGGTGG AATTCGGCTT | (SEQ ID NO:3) |
| GTCACTAGGA GAACATTTGT GTTAATTGCA | |
| CTGTGCTCTG TCAAGGAAAC TTTGATTTAT | |
| AGCTGGGGTG CACAAATAAT GGTTGCCGGT | |
| CGCACATGGA TTCGGTAGAA CTTTGCCTTC | |
| CTGAATCTTT TTCCCTGCAC TACGAGGAAG | |
| AGCTTCTCTG CAGAATGTCA AACAAAGATC | |
| GACACATTGA TTCCAGCTGT TCGTCCTTCA | |
| TCAAGACGGA ACCTTCCAGC CCAGCCTCCC | |
| TGACGGACAG CGTCAACCAC CACAGCCCTG | |
| GTGGCTCTTC AGACGCCAGT GGGAGCTACA | |
| GTTCAACCAT GAATGGCCAT CAGAACGGAC | |
| TTGACTCGCC ACCTCTCTAC CCTTCTGCTC | |
| CTATCCTGGG AGGTAGTGGG CCTGTCAGGA | |
| AACTGTATGA TGACTGCTCC AGCACCATTG | |
| TTGAAGATCC CCAGACCAAG TGTGAATACA | |
| TGCTCAACTC GATGCCCAAG AGACTGTGTT | |
| TAGTGTGTGG TGACATCGCT TCTGGGTACC | |
| ACTATGGGGT AGCATCATGT GAAGCCTGCA | |
| AGGCATTCTT CAAGAGGACA ATTCAAGGCA | |
| ATATAGAATA CAGCTGCCCT GCCACGAATG | |
| AATGTGAAAT CACAAAGCGC AGACGTAAAT | |
| CCTGCCAGGC TTGCCGCTTC ATGAAGTGTT | |
| TAAAAGTGGG CATGCTGAAA GAAGGGGTGC | |
| GTCTTGACAG AGTACGTGGA GGTCGGCAGA | |
| AGTACAAGCG CAGGATAGAT GCGGAGAACA | |
| GCCCATACCT GAACCCTCAG CTGGTTCAGC | |
| CAGCCAAAAA GCCATATAAC AAGATTGTCT | |
| CACATTTGTT GGTGGCTGAA CCGGAGAAGA | |
| TCTATGCCAT GCCTGACCCT ACTGTCCCCG | |
| ACAGTGACAT CAAAGCCCTC ACTACACTGT | |
| GTGACTTGGC CGACCGAGAG TTGGTGGTTA | |
| TCATTGGATG GGCGAAGCAT ATTCCAGGCT | |
| TCTCCACGCT GTCCCTGGCG GACCAGATGA | |
| GCCTTCTGCA GAGTGCTTGG ATGGAAATTT | |
| TGATCCTTGG TGTCGTATAC CGGTCTCTTT | |
| CATTTGAGGA TGAACTTGTC TATGCAGACG | |
| ATTATATAAT GGACGAAGAC CAGTCCAAAT | |
| TAGCAGGCCT TCTTGATCTA AATAATGCTA | |
| TCCTGCAGCT GGTAAAGAAA TACAAGAGCA | |
| TGAAGCTGGA AAAAGAAGAA TTTGTCACCC | |
| TCAAAGCTAT AGCTCTTGCT AATTCAGACT | |
| CCATGCACAT AGAAGATGTT GAAGCCGTTC | |
| AGAAGCTTCA GGATGTCTTA CATGAAGCGC | |
| TGCAGGATTA TGAAGCTGGC CAGCACATGG | |
| AAGACCCTCG TCGAGCTGGC AAGATGCTGA | |
| TGACACTGCC ACTCCTGAGG CAGACCTCTA | |
| CCAAGGCCGT GCAGCATTTC TACAACATCA | |
| AACTAGAAGG CAAAGTCCCA ATGCACAAAC | |
| TTTTTTTGGA AATGTTGGAG GCCAAGGTCT | |
| GACTAAAAGC TCCCTGGGCC TTCCCATCCT | |
| TCATGTTGAA AAAGGGAAAA TAAACCCAAG | |
| AGTGATGTCG AAGAAACTTA GAGTTTAGTT | |
| AACAACATCA AAAATCAACA GACTGCACTG | |
| ATAATTTAGC AGCAAGACTA TGAAGCAGCT | |
| TTCAGATTCC TCCATAGGTT CCTGATGAGT | |
| TCTTTCTACT TTCTCCATCA TCTTCTTTCC | |
| TCTTTCTTCC CACATTTCTC TTTCTCTTTA | |
| TTTTTTCTCC TTTTCTTCTT TCACCTCCCT | |
| TATTTCTTTG CTTCTTTCAT TCCTAGTTCC | |
| CATTCTCCTT TATTTTCTTC CCGTCTGCCT | |
| GCCTTCTTTC TTTTCTTTAC CTACTCTCAT | |
| TCCTCTCTTT TCTCATCCTT CCCCTTTTTT | |
| CTAAATTTGA AATAGCTTTA GTTTAAAAAA | |
| AAAAATCCTC CCTTCCCCCT TTCCTTTCCC | |
| TTTCTTTCCT TTTTCCCTTT CCTTTTCCCT | |
| TTCCTTTCCT TTCCTCTTGA CCTTCTTTCC | |
| ATCTTTCTTT TTCTTCCTTC TGCTGCTGAA | |
| CTTTTAAAAG AGGTCTCTAA CTGAAGAGAG | |
| ATGGAAGCCA GCCCTGCCAA AGGATGGAGA | |
| TCCATAATAT GGATGCCAGT GAACTTATTG | |
| TGAACCATAC CGTCCCCAAT GACTAAGGAA | |
| TCAAAGAGAG AGAACCAACG TTCCTAAAAG | |
| TACAGTGCAA CATATACAAA TTGACTGAGT | |
| GCAGTATTAG ATTTCATGGG AGCAGCCTCT | |
| AATTAGACAA CTTAAGCAAC GTTGCATCGG | |
| CTGCTTCTTA TCATTGCTTT TCCATCTAGA | |
| TCAGTTACAG CCATTTGATT CCTTAATTGT | |
| TTTTTCAAGT CTTCCAGGTA TTTGTTAGTT | |
| TAGCTACTAT GTAACTTTTT CAGGGAATAG | |
| TTTAAGCTTT ATTCATTCAT GCAATACTAA | |
| AGAGAAATAA GAATACTGCA ATTTTGTGCT | |
| GGCTTTGAAC AATTACGAAC AATAATGAAG | |
| GACAAATGAA TCCTGAAGGA AGATTTTTAA | |
| AAATGTTTTG TTTCTTCTTA CAAATGGAGA | |
| TTTTTTTGTA CCAGCTTTAC CACTTTTCAG | |
| CCATTTATTA ATATGGGAAT TTAACTTACT | |
| CAAGCAATAG TTGAAGGGAA GGTGCATATT | |
| ATCACGGATG CAATTTATGT TGTGTGCCAG | |
| TCTGGTCCCA AACATCAATT TCTTAACATG | |
| AGCTCCAGTT TACCTAAATG TTCACTGACA | |
| CAAAGGATGA GATTACACCT ACAGTGACTC | |
| TGAGTAGTCA CATATATAAG CACTGCACAT | |
| GAGATATAGA TCCGTAGAAT TGTCAGGAGT | |
| GCACCTCTCT ACTTGGGAGG TACAATTGCC | |
| ATATGATTTC TAGCTGCCAT GGTGGTTAGG | |
| AATGTGATAC TGCCTGTTTG CAAAGTCACA | |
| GACCTTGCCT CAGAAGGAGC TGTGAGCCAG | |
| TATTCATTTA AGAGAATTCC ACCACACTGG | |
| CGGCCCGCGC TTGAT. | |

39. A DNA molecule of claim 38 which consists of nucleotide 126 to about nucleotide 1382 of SEQ ID NO:3.

40. An expression vector for expressing a human nNR2 protein wherein said expression vector comprises a DNA molecule of claim 38.

41. An expression vector for expressing a human nNR2 protein wherein said expression vector comprises a DNA molecule of claim 39.

42. A host cell which expresses a recombinant human nNR2 protein wherein said host cell contains the expression vector of claim 40.

43. A host cell which expresses a recombinant human nNR2 protein wherein said host cell contains the expression vector of claim 41.

44. A process for expressing a human nNR2 protein in a recombinant host cell, comprising:
(a) transfecting the expression vector of claim 40 into a suitable host cell; and,
(b) culturing the host cells of step (a) under conditions which allow expression of said the human nNR2 protein from said expression vector.


SEQUENCE LISTING (30 sequences)

SEQ ID NO: 1 - DNA, 2807 bp, Human
gaatatgatgaccctaatgcaacaatatctaacatactatccgagcttcggtcatttggaagaactgcagattttcctccttcaaaattaaagtcaggttatggagaacatgtatgctatgttcttgattgcttcgctgaagaagcattgaaatatattggtttcacctggaaaaggccaatatacccagtagaagaattagaagaagaaagcgttgcagaagatgatgcagaattaacattaaataaagtggatgaagaatttgtggaagaagagacagataatgaagaaaactttattgatctcaacgttttaaaggcccagacatatcacttggatatgaacgagactgccaaacaagaagatattttggaatccacaacagatgctgcagaatggagcctagaagtggaacgtgtactaccgcaactgaaagtcacgattaggactgacaataaggattggagaatccatgttgaccaaatgcaccagcacagaagtggaattgaatctgctctaaaggagaccaagggatttttggacaaactccataatgaaattactaggactttggaaaagatcagcagccgagaaaagtacatcaacaatcagccgggagcccatggagcactgtcctcagagatgcgcaggttaggctcactgtctaggccaggcccaccttagtcactgtggactggcaatggaagctcttcctggacacacctgccctagccctcaccctggggtggaagagaaatgagcttggcttgcaactcagaccattccacggaggcatcctccccttccctgggctggtgaataaaagtttcctgaggtcaaggacttccttttccctgccaaaatggtgtccagaactttgaggccagaggtgatccagtgatttgggagctgcaggtcacacaggctgctcagagggctgctgaacaggatgtcctcggacgacaggcacctgggctccagctgcggctccttcatcaagactgagccgtccagcccgtcctcgggcatagatgccctcagccaccacagccccagtggctcgtccgacgccagcggcggctttggcctggccctgggcacccacgccaacggtctggactcgccacccatgtttgcaggcgccgggctgggaggcaccccatgccgcaagagctacgaggactgtgccagcggcatcatggaggactcggccatcaagtgcgagtacatgctcaacgccatccccaagcgcctgtgcctcgtgtgcggggacattgcctctggctaccactacggcgtggcctcctgcgaggcttgcaaggccttcttcaagaggactatccaagggaacattgagtacagctgcccggccaccaacgagtgcgagatcaccaaacggaggcgcaagtcctgccaggcctgccgcttcatgaaatgcctcaaagtggggatgctgaaggaaggtgtgcgccttgatcgagtgcgtggaggccgtcagaaatacaagcgacggctggactcagagagcagcccatacctgagcttacaaatttctccacctgctaaaaagccattgaccaagattgtctcatacctactggtggctgagccggacaagctctatgccatgcctccccctggtatgcctgagggggacatcaaggccctgaccactctctgtgacctggcagaccgagagcttgtggtcatcattggctgggccaagcacatcccaggcttctcaagcctctccctgggggaccagatgagcctgctgcagagtgcctggatggaaatcctcatcctgggcatcgtgtaccgctcgctgccctacgacgacaagctggtgtacgctgaggactacatcatggatgaggagcactcccgcctcgcggggctgctggagctctaccgggccatcctgcagctggtacgcaggtacaagaagctcaaggtggagaaggaggagtttgtgacgctcaaggccctggccctcgccaactccgattccatgtacatcgaggatctagaggctgtccagaagctgcaggacctgctgcacgaggcactgcaggactacgagctgagccagcgccatgaggagccctggaggacgggcaagctgctgctgacactgccgctgctgcggcagacggccgccaaggccgtgcagcacttctatagcgtcaaactgcagggcaaagtgcccatgcacaaactcttcctggagatgctggaggccaaggcctgggccagggctgactcccttcaggagtggaggccactggagcaagtgccctctcccctccaccgagccaccaagaggcagcatgtgcatttcctaactcccttgccccctcccccatctgtggcctgggtgggcactgctcaggctggataccacctggaggttttccttccgcagagggcaggttggccaagagcagcttagaggatctcccaaggatgaaagaatgtcaagccatgatggaaaatgccccttccaatcagctgccttcacaagcagggatcagagcaactccccggggatccccaatccacgcccttctagtccaacccccctcaatgagagaggcaggcagatctcacccagcactaggacaccaggaggccagggaaagcatctctggctcaccatgtaacatctggcttggagcaagtgggtgttctgcacaccaggcagctgcacctcactggatctagtgttgctgcgagtgacctcacttcagagcccctctagcagagtggggcggaagtcctgatggttggtgtccatgaggtggaag

SEQ ID NO: 2 - protein, 500 aa, Human
MSSDDRHLGSSCGSFIKTEPSSPSSGIDALSHHSPSGSSDASGGFGLALGTHANGLDSPPMFAGAGLGGTPCRKSYEDCASGIMEDSAIKCEYMLNAIPKRLCLVCGDIASGYHYGVASCEACKAFFKRTIQGNIEYSCPATNECEITKRRRKSCQACRFMKCLKVGMLKEGVRLDRVRGGRQKYKRRLDSESSPYLSLQISPPAKKPLTKIVSYLLVAEPDKLYAMPPPGMPEGDIKALTTLCDLADRELVVIIGWAKHIPGFSSLSLGDQMSLLQSAWMEILILGIVYRSLPYDDKLVYAEDYIMDEEHSRLAGLLELYRAILQLVRRYKKLKVEKEEFVTLKALALANSDSMYIEDLEAVQKLQDLLHEALQDYELSQRHEEPWRTGKLLLTLPLLRQTAAKAVQHFYSVKLQGKVPMHKLFLEMLEAKAWARADSLQEWRPLEQVPSPLHRATKRQHVHFLTPLPPPPSVAWVGTAQAGYHLEVFLPQRAGWPRAA

SEQ ID NO: 3 - DNA, 2985 bp, Human
gcgggccgccagtgtggtggaattcggcttgtcactaggagaacatttgtgttaattgcactgtgctctgtcaaggaaactttgatttatagctggggtgcacaaataatggttgccggtcgcacatggattcggtagaactttgccttcctgaatctttttccctgcactacgaggaagagcttctctgcagaatgtcaaacaaagatcgacacattgattccagctgttcgtccttcatcaagacggaaccttccagcccagcctccctgacggacagcgtcaaccaccacagccctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggcaatatagaatacagctgccctgccacgaatgaatgtgaaatcacaaagcgcagacgtaaatcctgccaggcttgccgcttcatgaagtgtttaaaagtgggcatgctgaaagaaggggtgcgtcttgacagagtacgtggaggtcggcagaagtacaagcgcaggatagatgcggagaacagcccatacctgaaccctcagctggttcagccagccaaaaagccatataacaagattgtctcacatttgttggtggctgaaccggagaagatctatgccatgcctgaccctactgtccccgacagtgacatcaaagccctcactacactgtgtgacttggccgaccgagagttggtggttatcattggatgggcgaagcatattccaggcttctccacgctgtccctggcggaccagatgagccttctgcagagtgcttggatggaaattttgatccttggtgtcgtataccggtctctttcatttgaggatgaacttgtctatgcagacgattatataatggacgaagaccagtccaaattagcaggccttcttgatctaaataatgctatcctgcagctggtaaagaaatacaagagcatgaagctggaaaaagaagaatttgtcaccctcaaagctatagctcttgctaattcagactccatgcacatagaagatgttgaagccgttcagaagcttcaggatgtcttacatgaagcgctgcaggattatgaagctggccagcacatggaagaccctcgtcgagctggcaagatgctgatgacactgccactcctgaggcagacctctaccaaggccgtgcagcatttctacaacatcaaactagaaggcaaagtcccaatgcacaaactttttttggaaatgttggaggccaaggtctgactaaaagctccctgggccttcccatccttcatgttgaaaaagggaaaataaacccaagagtgatgtcgaagaaacttagagtttagttaacaacatcaaaaatcaacagactgcactgataatttagcagcaagactatgaagcagctttcagattcctccataggttcctgatgagttctttctactttctccatcatcttctttcctctttcttcccacatttctctttctctttattttttctccttttcttctttcacctcccttatttctttgcttctttcattcctagttcccattctcctttattttcttcccgtctgcctgccttctttcttttctttacctactctcattcctctcttttctcatccttccccttttttctaaatttgaaatagctttagtttaaaaaaaaaaatcctcccttccccctttcctttccctttctttcctttttccctttccttttccctttcctttcctttcctcttgaccttctttccatctttctttttcttccttctgctgctgaacttttaaaagaggtctctaactgaagagagatggaagccagccctgccaaaggatggagatccataatatggatgccagtgaacttattgtgaaccataccgtccccaatgactaaggaatcaaagagagagaaccaacgttcctaaaagtacagtgcaacatatacaaattgactgagtgcagtattagatttcatgggagcagcctctaattagacaacttaagcaacgttgcatcggctgcttcttatcattgcttttccatctagatcagttacagccatttgattccttaattgttttttcaagtcttccaggtatttgttagtttagctactatgtaactttttcagggaatagtttaagctttattcattcatgcaatactaaagagaaataagaatactgcaattttgtgctggctttgaacaattacgaacaataatgaaggacaaatgaatcctgaaggaagatttttaaaaatgttttgtttcttcttacaaatggagatttttttgtaccagctttaccacttttcagccatttattaatatgggaatttaacttactcaagcaatagttgaagggaaggtgcatattatcacggatgcaatttatgttgtgtgccagtctggtcccaaacatcaatttcttaacatgagctccagtttacctaaatgttcactgacacaaaggatgagattacacctacagtgactctgagtagtcacatatataagcactgcacatgagatatagatccgtagaattgtcaggagtgcacctctctacttgggaggtacaattgccatatgatttctagctgccatggtggttaggaatgtgatactgcctgtttgcaaagtcacagaccttgcctcagaaggagctgtgagccagtattcatttaagagaattccaccacactggcggcccgcgcttgat

SEQ ID NO: 4 - protein, 458 aa, Human
MDSVELCLPESFSLHYEEELLCRMSNKDRHIDSSCSSFIKTEPSSPASLTDSVNHHSPGGSSDASGSYSSTMNGHQNGLDSPPLYPSAPILGGSGPVRKLYDDCSSTIVEDPQTKCEYMLNSMPKRLCLVCGDIASGYHYGVASCEACKAFFKRTIQGNIEYSCPATNECEITKRRRKSCQACRFMKCLKVGMLKEGVRLDRVRGGRQKYKRRIDAENSPYLNPQLVQPAKKPYNKIVSHLLVAEPEKIYAMPDPTVPDSDIKALTTLCDLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDSMHIEDVEAVQKLQDVLHEALQDYEAGQHMEDPRRAGKMLMTLPLLRQTSTKAVQHFYNIKLEGKVPMHKLFLEMLEAKV

SEQ ID NO: 5 - DNA, 2987 bp, Human
gcgggccgccagtgtggtggaattcggcttgtcactaggagaacatttgtgttaattgcactgtgctctgtcaaggaaactttgatttatagctggggtgcacaaataatggttgccggtcgcacatggattcggtagaactttgccttcctgaatctttttccctgcactacgaggaagagcttctctgcagaatgtcaaacaaagatcgacacattgattccagctgttcgtccttcatcaagacggaaccttccagcccagcctccctgacggacagcgtcaaccaccacagccctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggcaatatagaatacagctgccctgccacgaatgaatgtgaaatcacaaagcgcagacgtaaatcctgccaggcttgccgcttcatgaagtgtttaaaagtgggcatgctgaaagaaggggtgcgtcttgacagagtacgtggaggtcggcagaagtacaagcgcaggatagatgcggagaacagcccatacctgaaccctcagctggttcagccagccaaaaagccatataacaagattgtctcacatttgttggtggctgaaccggagaagatctatgccatgcctgaccctactgtccccgacagtgacatcaaagccctcactacactgtgtgacttggccgaccgagagttggtggttatcattggatgggcgaagcatattccaggcttctccacgctgtccctggcggaccagatgagccttctgcagagtgcttggatggaaattttgatccttggtgtcgtataccggtctctttcatttgaggatgaacttgtctatgcagacgattatataatggacgaagaccagtccaaattagcaggccttcttgatctaaataatgctatcctgcagctggtaaagaaatacaagagcatgaagctggaaaaagaagaatttgtcaccctcaaagctatagctcttgctaattcagactccatgcacatagaagatgttgaagccgttcagaagcttcaggatgtcttacatgaagcgctgcaggattatgaagctggccagcacatggagaagaccctcgtcgagctggcaagatgctgatgacactgccactcctgaggcagacctctaccaaggccgtgcagcatttctacaacatcaaactagaaggcaaagtcccaatgcacaaactttttttggaaatgttggaggccaaggtctgactaaaagctccctgggccttcccatccttcatgttgaaaaagggaaaataaacccaagagtgatgtcgaagaaacttagagtttagttaacaacatcaaaaatcaacagactgcactgataatttagcagcaagactatgaagcagctttcagattcctccataggttcctgatgagttctttctactttctccatcatcttctttcctctttcttcccacatttctctttctctttattttttctccttttcttctttcacctcccttatttctttgcttctttcattcctagttcccattctcctttattttcttcccgtctgcctgccttctttcttttctttacctactctcattcctctcttttctcatccttccccttttttctaaatttgaaatagctttagtttaaaaaaaaaaatcctcccttccccctttcctttccctttctttcctttttccctttccttttccctttcctttcctttcctcttgaccttctttccatctttctttttcttccttctgctgctgaacttttaaaagaggtctctaactgaagagagatggaagccagccctgccaaaggatggagatccataatatggatgccagtgaacttattgtgaaccataccgtccccaatgactaaggaatcaaagagagagaaccaacgttcctaaaagtacagtgcaacatatacaaattgactgagtgcagtattagatttcatgggagcagcctctaattagacaacttaagcaacgttgcatcggctgcttcttatcattgcttttccatctagatcagttacagccatttgattccttaattgttttttcaagtcttccaggtatttgttagtttagctactatgtaactttttcagggaatagtttaagctttattcattcatgcaatactaaagagaaataagaatactgcaattttgtgctggctttgaacaattacgaacaataatgaaggacaaatgaatcctgaaggaagatttttaaaaatgttttgtttcttcttacaaatggagatttttttgtaccagctttaccacttttcagccatttattaatatgggaatttaacttactcaagcaatagttgaagggaaggtgcatattatcacggatgcaatttatgttgtgtgccagtctggtcccaaacatcaatttcttaacatgagctccagtttacctaaatgttcactgacacaaaggatgagattacacctacagtgactctgagtagtcacatatataagcactgcacatgagatatagatccgtagaattgtcaggagtgcacctctctacttgggaggtacaattgccatatgatttctagctgccatggtggttaggaatgtgatactgcctgtttgcaaagtcacagaccttgcctcagaaggagctgtgagccagtattcatttaagagaattccaccacactggcggcccgcgcttgat

SEQ ID NO: 6 - protein, 418 aa, Human
MDSVELCLPESFSLHYEEELLCRMSNKDRHIDSSCSSFIKTEPSSPASLTDSVNHHSPGGSSDASGSYSSTMNGHQNGLDSPPLYPSAPILGGSGPVRKLYDDCSSTIVEDPQTKCEYMLNSMPKRLCLVCGDIASGYHYGVASCEACKAFFKRTIQGNIEYSCPATNECEITKRRRKSCQACRFMKCLKVGMLKEGVRLDRVRGGRQKYKRRIDAENSPYLNPQLVQPAKKPYNKIVSHLLVAEPEKIYAMPDPTVPDSDIKALTTLCDLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDSMHIEDVEAVQKLQDVLHEALQDYEAGQHMEKTLVELARC

SEQ ID NO: 7 - DNA, 403 bp, Human
  misc_feature (1)...(403): n = A,T,C or G
ctttttaggaggtggagaaatttgtaagctcaggtatgggctgctctctgagtccagccgtcgcttgtatttctgacggcctccacgcactcgatcaaggcgcacaccttccttcagcatccccactttgaggcatttcatgaagcggcaggcctggcaggacttgcgcctccgtttggtgatctcgcactcgttggtggccgggcagctgtactcaatgttcccttggatagtcctcttgaagaaggccttgcaagcctcgcaggaggcccacgcgtnagtggtagccagagnaaatgtccccgcacacgaggcacaggcgcttggggatggcgttgagcatgttacttcgcacttggatgggccgagtcctccatggatggccgctggcaacagttcctcg

SEQ ID NO: 8 - DNA, 622 bp, Human
  misc_feature (1)...(622): n = A,T,C or G
cnnnnnnnnnnnnttttnntgcctaaagtggtacccngaagngatgtcaccacacactaaacacagtctcttgggcatcgagttgagcatgtattcacacttggtctggggatcttcaacaatggtgctggagcagtcatcatacagtttcctgacaggcccactacctcccaggataggagcagaagggtagagaggtggcgagtcaagtccgttctgatggccattcatggttgaactgtagctcccactggcgtctgaagagccaccagggctgtggtggttgacgctgtccgtcagggaggctgggctggaaggttccgtcttgatgaaggacgaacagctggaatcaatgtgtcgatctttgtttggacattctgcagagaagctcttcctccgtngtgcagggaaaaagattcaggaaggcaaagttcttcccgaatccatgtgcgaccggaaaccattatttgngcaccccagctattaatcaaagttccttgacagagacagggcaattacanaatgtctcctntnggggatcaactgttcngtattnnnnnnnnnnnnnnnnnnnnnnnnnnnnnnnnnnnnnnnnnnnnnnnnnnnnnnnnnnnnnnnnntt

SEQ ID NO: 9 - DNA, 22 bp, Artificial Sequence
  oligonucleotide
tgagtccagccgtcgcttgtat

SEQ ID NO: 10 - DNA, 20 bp, Artificial Sequence
  oligonucleotide
tgcaagcctcgcaggaggcc

SEQ ID NO: 11 - DNA, 22 bp, Artificial Sequence
  oligonucleotide
ggccttcttcaagaggactatc

SEQ ID NO: 12 - DNA, 21 bp, Artificial Sequence
  oligonucleotide
aaagatcgacacattgattcc

SEQ ID NO: 13 - DNA, 20 bp, Artificial Sequence
  oligonucleotide
gacttgactcgccacctctc

SEQ ID NO: 14 - DNA, 21 bp, Artificial Sequence
  oligonucleotide
gttctgatggccattcatggt

SEQ ID NO: 15 - DNA, 21 bp, Artificial Sequence
  oligonucleotide
gaatatgatgaccctaatgca

SEQ ID NO: 16 - DNA, 21 bp, Artificial Sequence
  oligonucleotide
cttccacctcatggacaccaa

SEQ ID NO: 17 - DNA, 20 bp, Artificial Sequence
  oligonucleotide
gttaattgcactgtgctctg

SEQ ID NO: 18 - DNA, 20 bp, Artificial Sequence
  oligonucleotide
agtgtggtggaattctctta

SEQ ID NO: 19 - DNA, 21 bp, Artificial Sequence
  oligonucleotide
tctagtgttgctgcgagtgac

SEQ ID NO: 20 - DNA, 21 bp, Artificial Sequence
  oligonucleotide
cttccacctcatggacaccaa

SEQ ID NO: 21 - DNA, 20 bp, Artificial Sequence
  oligonucleotide
gtctgactaaaagctccctg

SEQ ID NO: 22 - DNA, 21 bp, Artificial Sequence
  oligonucleotide
gaagatgatggagaaagtaga

SEQ ID NO: 23 - DNA, 20 bp, Artificial Sequence
  oligonucleotide
cattccacggaggcatcctc

SEQ ID NO: 24 - DNA, 20 bp, Artificial Sequence
  oligonucleotide
ccaaggccgtgcagcacttc

SEQ ID NO: 25 - DNA, 21 bp, Artificial Sequence
  oligonucleotide
gacagcctctagatcctcgat

SEQ ID NO: 26 - DNA, 21 bp, Artificial Sequence
  oligonucleotide
atcatggcttgacattctttc

SEQ ID NO: 27 - DNA, 19 bp, Artificial Sequence
  oligonucleotide
agctcttgctaattcagac

SEQ ID NO: 28 - DNA, 21 bp, Artificial Sequence
  oligonucleotide
tcaacatgaaggatgggaagg

SEQ ID NO: 29 - DNA, 2807 bp, Human
cttatactactgggattacgttgttatagattgtatgataggctcgaagccagtaaaccttcttgacgtctaaaaggaggaagttttaatttcagtccaatacctcttgtacatacgatacaagaactaacgaagcgacttcttcgtaactttatataaccaaagtggaccttttccggttatatgggtcatcttcttaatcttcttctttcgcaacgtcttctactacgtcttaattgtaatttatttcacctacttcttaaacaccttcttctctgtctattacttcttttgaaataactagagttgcaaaatttccgggtctgtatagtgaacctatacttgctctgacggtttgttcttctataaaaccttaggtgttgtctacgacgtcttacctcggatcttcaccttgcacatgatggcgttgactttcagtgctaatcctgactgttattcctaacctcttaggtacaactggtttacgtggtcgtgtcttcaccttaacttagacgagatttcctctggttccctaaaaacctgtttgaggtattactttaatgatcctgaaaccttttctagtcgtcggctcttttcatgtagttgttagtcggccctcgggtacctcgtgacaggagtctctacgcgtccaatccgagtgacagatccggtccgggtggaatcagtgacacctgaccgttaccttcgagaaggacctgtgtggacgggatcgggagtgggaccccaccttctctttactcgaaccgaacgttgagtctggtaaggtgcctccgtaggaggggaagggacccgaccacttattttcaaaggactccagttcctgaaggaaaagggacggttttaccacaggtcttgaaactccggtctccactaggtcactaaaccctcgacgtccagtgtgtccgacgagtctcccgacgacttgtcctacaggagcctgctgtccgtggacccgaggtcgacgccgaggaagtagttctgactcggcaggtcgggcaggagcccgtatctacgggagtcggtggtgtcggggtcaccgagcaggctgcggtcgccgccgaaaccggaccgggacccgtgggtgcggttgccagacctgagcggtgggtacaaacgtccgcggcccgaccctccgtggggtacggcgttctcgatgctcctgacacggtcgccgtagtacctcctgagccggtagttcacgctcatgtacgagttgcggtaggggttcgcggacacggagcacacgcccctgtaacggagaccgatggtgatgccgcaccggaggacgctccgaacgttccggaagaagttctcctgataggttcccttgtaactcatgtcgacgggccggtggttgctcacgctctagtggtttgcctccgcgttcaggacggtccggacggcgaagtactttacggagtttcacccctacgacttccttccacacgcggaactagctcacgcacctccggcagtctttatgttcgctgccgacctgagtctctcgtcgggtatggactcgaatgtttaaagaggtggacgatttttcggtaactggttctaacagagtatggatgaccaccgactcggcctgttcgagatacggtacggagggggaccatacggactccccctgtagttccgggactggtgagagacactggaccgtctggctctcgaacaccagtagtaaccgacccggttcgtgtagggtccgaagagttcggagagggaccccctggtctactcggacgacgtctcacggacctacctttaggagtaggacccgtagcacatggcgagcgacgggatgctgctgttcgaccacatgcgactcctgatgtagtacctactcctcgtgagggcggagcgccccgacgacctcgagatggcccggtaggacgtcgaccatgcgtccatgttcttcgagttccacctcttcctcctcaaacactgcgagttccgggaccgggagcggttgaggctaaggtacatgtagctcctagatctccgacaggtcttcgacgtcctggacgacgtgctccgtgacgtcctgatgctcgactcggtcgcggtactcctcgggacctcctgcccgttcgacgacgactgtgacggcgacgacgccgtctgccggcggttccggcacgtcgtgaagatatcgcagtttgacgtcccgtttcacgggtacgtgtttgagaaggacctctacgacctccggttccggacccggtcccgactgagggaagtcctcacctccggtgacctcgttcacgggagaggggaggtggctcggtggttctccgtcgtacacgtaaaggattgagggaacgggggagggggtagacaccggacccacccgtgacgagtccgacctatggtggacctccaaaaggaaggcgtctcccgtccaaccggttctcgtcgaatctcctagagggttcctactttcttacagttcggtactaccttttacggggaaggttagtcgacggaagtgttcgtccctagtctcgttgaggggcccctaggggttaggtgcgggaagatcaggttggggggagttactctctccgtccgtctagagtgggtcgtgatcctgtggtcctccggtccctttcgtagagaccgagtggtacattgtagaccgaacctcgttcacccacaagacgtgtggtccgtcgacgtggagtgacctagatcacaacgacgctcactggagtgaagtctcggggagatcgtctcaccccgccttcaggactaccaaccacaggtactccaccttc

SEQ ID NO: 30 - DNA, 2985 bp, Human
cgcccggcggtcacaccaccttaagccgaacagtgatcctcttgtaaacacaattaacgtgacacgagacagttcctttgaaactaaatatcgaccccacgtgtttattaccaacggccagcgtgtacctaagccatcttgaaacggaaggacttagaaaaagggacgtgatgctccttctcgaagagacgtcttacagtttgtttctagctgtgtaactaaggtcgacaagcaggaagtagttctgccttggaaggtcgggtcggagggactgcctgtcgcagttggtggtgtcgggaccaccgagaagtctgcggtcaccctcgatgtcaagttggtacttaccggtagtcttgcctgaactgagcggtggagagatgggaagacgaggataggaccctccatcacccggacagtcctttgacatactactgacgaggtcgtggtaacaacttctaggggtctggttcacacttatgtacgagttgagctacgggttctctgacacaaatcacacaccactgtagcgaagacccatggtgataccccatcgtagtacacttcggacgttccgtaagaagttctcctgttaagttccgttatatcttatgtcgacgggacggtgcttacttacactttagtgtttcgcgtctgcatttaggacggtccgaacggcgaagtacttcacaaattttcacccgtacgactttcttccccacgcagaactgtctcatgcacctccagccgtcttcatgttcgcgtcctatctacgcctcttgtcgggtatggacttgggagtcgaccaagtcggtcggtttttcggtatattgttctaacagagtgtaaacaaccaccgacttggcctcttctagatacggtacggactgggatgacaggggctgtcactgtagtttcgggagtgatgtgacacactgaaccggctggctctcaaccaccaatagtaacctacccgcttcgtataaggtccgaagaggtgcgacagggaccgcctggtctactcggaagacgtctcacgaacctacctttaaaactaggaaccacagcatatggccagagaaagtaaactcctacttgaacagatacgtctgctaatatattacctgcttctggtcaggtttaatcgtccggaagaactagatttattacgataggacgtcgaccatttctttatgttctcgtacttcgacctttttcttcttaaacagtgggagtttcgatatcgagaacgattaagtctgaggtacgtgtatcttctacaacttcggcaagtcttcgaagtcctacagaatgtacttcgcgacgtcctaatacttcgaccggtcgtgtaccttctgggagcagctcgaccgttctacgactactgtgacggtgaggactccgtctggagatggttccggcacgtcgtaaagatgttgtagtttgatcttccgtttcagggttacgtgtttgaaaaaaacctttacaacctccggttccagactgattttcgagggacccggaagggtaggaagtacaactttttcccttttatttgggttctcactacagcttctttgaatctcaaatcaattgttgtagtttttagttgtctgacgtgactattaaatcgtcgttctgatacttcgtcgaaagtctaaggaggtatccaaggactactcaagaaagatgaaagaggtagtagaagaaaggagaaagaagggtgtaaagagaaagagaaataaaaaagaggaaaagaagaaagtggagggaataaagaaacgaagaaagtaaggatcaagggtaagaggaaataaaagaagggcagacggacggaagaaagaaaagaaatggatgagagtaaggagagaaaagagtaggaaggggaaaaaagatttaaactttatcgaaatcaaatttttttttttaggagggaagggggaaaggaaagggaaagaaaggaaaaagggaaaggaaaagggaaaggaaaggaaaggagaactggaagaaaggtagaaagaaaaagaaggaagacgacgacttgaaaattttctccagagattgacttctctctaccttcggtcgggacggtttcctacctctaggtattatacctacggtcacttgaataacacttggtatggcaggggttactgattccttagtttctctctcttggttgcaaggattttcatgtcacgttgtatatgtttaactgactcacgtcataatctaaagtaccctcgtcggagattaatctgttgaattcgttgcaacgtagccgacgaagaatagtaacgaaaaggtagatctagtcaatgtcggtaaactaaggaattaacaaaaaagttcagaaggtccataaacaatcaaatcgatgatacattgaaaaagtcccttatcaaattcgaaataagtaagtacgttatgatttctctttattcttatgacgttaaaacacgaccgaaacttgttaatgcttgttattacttcctgtttacttaggacttccttctaaaaatttttacaaaacaaagaagaatgtttacctctaaaaaaacatggtcgaaatggtgaaaagtcggtaaataattatacccttaaattgaatgagttcgttatcaacttcccttccacgtataatagtgcctacgttaaatacaacacacggtcagaccagggtttgtagttaaagaattgtactcgaggtcaaatggatttacaagtgactgtgtttcctactctaatgtggatgtcactgagactcatcagtgtatatattcgtgacgtgtactctatatctaggcatcttaacagtcctcacgtggagagatgaaccctccatgttaacggtatactaaagatcgacggtaccaccaatccttacactatgacggacaaacgtttcagtgtctggaacggagtcttcctcgacactcggtcataagtaaattctcttaaggtggtgtgaccgccgggcgcgaacta